(19) United States
(12) Patent Application Publication
(10) Pub. No.: US 2017/0322228 A1
(43) Pub. Date: Nov. 9, 2017

(54) METHOD, KIT AND TEST STRIP FOR DETECTING KAWASAKI DISEASE

(71) Applicant: PUBLIC UNIVERSITY CORPORATION YOKOHAMA CITY UNIVERSITY, Yokohama-shi (JP)

(72) Inventors: Yayoi HORIUCHI, Yokohama-shi (JP); Masaaki Mori, Tokyo (JP); Hisashi Hirano, Yokohama-shi (JP); Shumpei Yokota, Yokohama-shi (JP); Yoko Saito, Yokohama-shi (JP); Mao Aketagawa, Yokohama-shi (JP)

(73) Assignee: PUBLIC UNIVERSITY CORPORATION YOKOHAMA CITY UNIVERSITY, Yokohama-shi (JP)

(21) Appl. No.: 15/659,815

(22) Filed: Jul. 26, 2017

Related U.S. Application Data
(63) Continuation-in-part of application No. PCT/JP2016/053940, filed on Feb. 10, 2016.

(30) Foreign Application Priority Data
Feb. 10, 2015 (JP) JP2015-024506
Aug. 9, 2016 (JP) JP2016-156241

(51) Int. Cl. G01N 33/68; G01N 33/74; G01N 33/573; G01N 30/00

(52) U.S. Cl. CPC G01N 33/6893; G01N 33/74; G01N 33/573; G01N 30/00; G01N 33/68; G01N 2800/328; G01N 2800/60

(57) ABSTRACT

The present invention provides methods in the context of Kawasaki disease including measuring the level of at least one of lipopolysaccharide binding protein, leucine-rich alpha-2-glycoprotein, angiotensinogen and retinol binding protein 4 in a sample derived from a subject.

This application is a continuation-in-part of International Application No. PCT/JP2016/053940, filed on Feb. 10, 2016, and is claiming the priority based on Japanese Patent Application No. 2016-156241, filed on Aug. 9, 2016.

### TECHNICAL FIELD

The present invention relates to a method, a kit and a test strip for testing for Kawasaki disease.

### BACKGROUND ART

Kawasaki disease is an acute, febrile, exanthematous disease seen mainly in infants of age four or below, and its major pathogenic condition is systemic vasculitis. Diagnosis of Kawasaki disease is performed based on appearance of a plurality of major symptoms (1. fever lasting for five or more days; 2. hyperemia of the both bulbar conjunctiva; 3. redness in the lips, strawberry tongue; 4. polymorphous rash; 5. hardness of the hand fingers/erythema of the hand fingers and the foot pads in the acute phase; membrane-like desquamation in the afebrile period following intervention; 6. non-purulent cervical lymphadenopathy) (“A Guide for Diagnosis of Kawasaki Disease”). Blood tests are also carried out to examine the increase of leucocytes, C reactive proteins and escape enzymes from hepatocytes, the enhancement of erythrocyte sedimentation rate, leucocyte fraction (neutrophil ratio) and the like. Further, confirmation of coronary artery lesions is carried out by two-dimensional echocardiography or cardioangiography.

Kawasaki disease cures spontaneously but if it is left untreated, 25-30% of patients will develop cardiovascular complications typified by coronary artery lesions. Therefore, it is very important to start treatment of Kawasaki disease at an early stage of its onset to thereby inhibit the inflammation. The febrile phase need be shortened as much as possible while at the same time, it is necessary to prevent the occurrence of cardiovascular complications. However, the cause and developmental mechanism of Kawasaki disease are still unknown. No specific diagnosis test exists for Kawasaki disease. Major symptoms vary from patient to patient, and there are a number of cases which do not meet the diagnostic criteria. Therefore, it is difficult to make a definitive diagnosis of Kawasaki disease quickly.

With respect to patents relating to diagnosis of Kawasaki disease, there are known a method in which the concentration of vascular endothelial growth factor (VEGF) in blood is measured (Patent Document No. 1: Japanese Unexamined Patent Publication No. Hei 11-6832); a method in which IgM to one or more super-antigens is measured (Patent Document No. 2: Japanese Unexamined Patent Publication No. Hei 3-139294); and an investigation into genetic polymorphism (Patent Document No. 3: Japanese Unexamined Patent Publication No. 2009-72193). However, none of these patents are actually used in clinical scenes.

### PRIOR ART LITERATURE

### Patent Documents

Patent Document No. 1: Japanese Unexamined Patent Publication No. Hei 11-6832
Patent Document No. 2: Japanese Unexamined Patent Publication No. Hei 3-139294

### Patent Document No. 3: Japanese Unexamined Patent Publication No. 2009-72193

### DISCLOSURE OF THE INVENTION

### Problem for Solution by the Invention

It is an object of the present invention to provide a method, a kit and a test strip for testing for Kawasaki disease quickly and simply.

### Means to Solve the Problem

As a result of intensive and extensive researches, the present inventors found that differences in the expression levels of lipopolysaccharide binding protein (LBP), leucine-rich alpha-2-glycoprotein 1 (LRG1), angiotensinogen (AGT) and retinol binding protein 4 (RBP4) in the sera of Kawasaki disease patients were statistically significant (p<0.0001) between the acute phase (the febrile period before intervention) and the recovery phase (the afebrile period following intervention). As regards LBP, LRG1, AGT and RBP4, specific antibodies thereto already exist and the amounts of these proteins in serum or blood can be determined with high sensitivity and in a simple way by using antigen-antibody reaction. In the present study, the inventors performed immunoblot analyses using an extremely small amount of serum (or whole blood) to find that the expressions of LBP, LRG1 and AGT were high whereas the expression of RBP4 was low in the acute phase of Kawasaki disease. Further, the present inventors have found that differences in the expression levels of serum lipopolysaccharide binding protein (LBP) and leucine-rich alpha-2-glycoprotein 1 (LRG1) between patients in the acute phase of Kawasaki disease (febrile period before intervention) and healthy subjects or patients with pediatric disease (autoimmune disease) are statistically significant (p<0.0001), and that it will be possible to diagnose Kawasaki disease specifically and with high sensitivity by setting appropriate cutoff values. The present invention has been achieved based on these findings.

Various embodiments of the invention include:
(1) A method of testing for Kawasaki disease, comprising measuring the level of at least one component selected from the group consisting of lipopolysaccharide binding protein, leucine-rich alpha-2-glycoprotein, angiotensinogen and retinol binding protein 4 in a sample derived from a subject.
(2) The method of (1) above, wherein it is judged that the subject is likely to be suffering from Kawasaki disease when the level of at least one component selected from the group consisting of lipopolysaccharide binding protein, leucine-rich alpha-2-glycoprotein and angiotensinogen in the sample derived from the subject is high whereas it is judged that the subject is less likely to be suffering from Kawasaki disease when the level of interest is low.
(3) The method of (1) above, wherein it is judged that the subject is likely to be suffering from Kawasaki disease when the level of retinol binding protein 4 in the sample derived from the subject is low whereas it is judged that the subject is less likely to be suffering from Kawasaki disease when the level of interest is high.
(4) The method of (1) above, wherein the subject is a patient receiving treatment of Kawasaki disease; the level of at least one component selected from the group consisting of lipopolysaccharide binding protein, leucine-rich alpha-2-glycoprotein and angiotensinogen in the sample derived from the subject is measured once or more than once at different points of time; and it is judged that the subject has been recovered from Kawasaki disease by the treatment when the level of interest is low or decreased whereas it is judged that the subject has not been recovered or not sufficiently recovered from Kawasaki disease by the treatment when the level of interest is high or not decreased.
(5) The method of (1) above, wherein the subject is a patient receiving treatment of Kawasaki disease; the level of retinol binding protein 4 in the sample derived from the subject is measured once or more than once at different points of time; and it is judged that the subject has been recovered from Kawasaki disease by the treatment when the level of interest is high or increased whereas it is judged that the subject has not been recovered or not sufficiently recovered from Kawasaki disease by the treatment when the level of interest is low or not increased.
(6) The method of any one of (1) to (5) above, wherein the sample derived from the subject is serum or whole blood.
(7) A test kit for Kawasaki disease, comprising at least one reagent selected from the group consisting of reagents capable of specifically detecting lipopolysaccharide binding protein, reagents capable of specifically detecting leucine-rich alpha-2-glycoprotein, reagents capable of specifically detecting angiotensinogen, and reagents capable of specifically detecting retinol binding protein 4.
(8) The kit of (7) above, wherein the reagent is an antibody.

The number of patients who are diagnosed as suffering from Kawasaki disease is around 10,000 per year. Patients with pediatric febrile diseases of unknown cause other than Kawasaki disease are also great in number and if these patients are diagnosed for Kawasaki disease by an initial screening test, the potential market scale will be large. Further, if the diagnosis is also applicable to judgement of the severity of Kawasaki disease, unnecessary use of expensive γ-globulin formulation as a remedy can be avoided, leading to another advantage of saving medical expenses.

(9) A method of testing for Kawasaki disease, comprising measuring the levels of lipopolysaccharide binding protein (LBP) and leucine-rich alpha-2-glycoprotein (LRG1) in a sample derived from a subject.
(10) The method of (9) above, wherein when the levels of LBP and LRG1 are respectively higher than specified values, it is judged that the subject is likely to be suffering from Kawasaki disease; and when the levels of LBP and LRG1 are respectively lower than the specified values, it is judged that the subject is less likely to be suffering from Kawasaki disease.
(11) The method of (9) or (10) above, wherein the sample derived from a patient is serum, whole blood or plasma.
(12) The method of any one of (9) to (11) above, wherein the measurement of the level of LBP and the level of LRG1 is performed by immunochromatography.
(13) A test strip for detecting Kawasaki disease by immunochromatography, comprising an anti-LBP antibody immobilized carrier and an anti-LRG1 antibody immobilized carrier.
(14) The test strip of (13) above, wherein the anti-LBP antibody immobilized carrier and the anti-LRG1 antibody immobilized carrier are included in the same test strip.
(15) The test strip of (13) above, wherein the anti-LBP antibody immobilized carrier and the anti-LRG1 antibody immobilized carrier are included in different test strips.
(16) In further, alternative or inclusive embodiments there is provided a method detecting a level of at least one component selected from the group consisting of lipopolysaccharide binding protein, leucine-rich alpha-2-glycoprotein, angiotensinogen and retinol binding protein 4, the method comprising measuring the level of the at least one component selected from the group consisting of lipopolysaccharide binding protein, leucine-rich alpha-2-glycoprotein, angiotensinogen and retinol binding protein 4 in a sample, and comparing the level of the at least one component selected from the group consisting of lipopolysaccharide binding protein, leucine-rich alpha-2-glycoprotein, angiotensinogen and retinol binding protein 4 measured in the sample to a control sample of the at least one component selected from the group consisting of lipopolysaccharide binding protein, leucine-rich alpha-2-glycoprotein, angiotensinogen and retinol binding protein 4 obtained from a patient suffering from Kawasaki disease, a control sample of the at least one component selected from the group consisting of lipopolysaccharide binding protein, leucine-rich alpha-2-glycoprotein, angiotensinogen and retinol binding protein 4 obtained from a patient not suffering from Kawasaki disease, or a combination of control samples thereof.
With regard to the method (16), the sample measured is a Kawasaki disease patient and may include a Kawasaki disease patient is receiving treatment for Kawasaki disease. The sample may be a serum sample or a whole blood sample. The control sample may be obtained from a patient suffering from Kawasaki disease and/or one that is not suffering from Kawasaki disease. If the patient sample is obtained from a patient suffering from Kawasaki disease, it may be preferred to obtain the sample between the acute phase and the recovery phase of the Kawasaki disease patient
The method (16) can include measuring comprises measuring with at least one antibody that specifically binds to the at least one component selected from the group consisting of lipopolysaccharide binding protein, leucine-rich alpha-2-glycoprotein, angiotensinogen and retinol binding protein 4.

The measuring of method (16) may be performed by immunochromatography, for instance with at least one antibody is immobilized on at least one solid support, e.g., at least one test strip that may comprise an anti-LBP antibody immobilized carrier, an anti-LRG1 antibody immobilized carrier, or both. In some aspects the measuring comprises two test strips, each comprising different antibodies or comprises a single test strip.

A further method (17) may include detecting a level of at least one component selected from the group consisting of lipopolysaccharide binding protein, leucine-rich alpha-2-glycoprotein, angiotensinogen and retinol binding protein 4, the method comprising comparing the level of the at least one component selected from the group consisting of lipopolysaccharide binding protein, leucine-rich alpha-2-glycoprotein, angiotensinogen and retinol binding protein 4 measured in a sample to a control sample of the at least one component selected from the group consisting of lipopolysaccharide binding protein, leucine-rich alpha-2-glycoprotein, angiotensinogen and retinol binding protein 4 obtained from a patient suffering from Kawasaki disease, a control sample of the at least one component selected from the group consisting of lipopolysaccharide binding protein, leucine-rich alpha-2-glycoprotein, angiotensinogen and retinol binding protein 4 obtained from a patient not suffering from Kawasaki disease, or a combination of control samples thereof.
A further method (18) may involve treating Kawasaki disease, comprising treating a subject in need thereof with a Kawasaki disease treatment, the subject having been identified as in need thereof by a method of diagnosing Kawasaki disease comprising measuring a level of at least one component selected from the group consisting of lipopolysaccharide binding protein, leucine-rich alpha-2-glycoprotein, angiotensinogen and retinol binding protein 4 and/or at least one of methods (16) and (17) above.

### Effect of the Invention

According to the present invention, a test method that imposes only a small burden on patients and which is perfomied in addition to the conventional diagnosis based on major symptoms enables Kawasaki disease to be diagnosed in a quick manner and at a very high probability. Further, according to the present invention, it is also possible to confirm therapeutic effects on Kawasaki disease.

The present specification encompasses the contents disclosed in the specification and/or the drawings of Japanese Patent Application No. 2015-024506 based on which the present patent application claims priority.

### BRIEF DESCRIPTION OF THE DRAWINGS

FIG. 1. Western blot images obtained with anti-LBP antibody. Samples assigned the same number represent that they are derived from the same patient; the sample from the acute phase and the sample from the recovery phase make a pair.

FIG. 2. Western blot images obtained with anti-LRG1 [ERR 12362] antibody. Samples assigned the same number represent that they are derived from the same patient; the sample from the acute phase and the sample from the recovery phase make a pair.

FIG. 3. Graphs obtained by quantifying and plotting the intensities of the bands detected by Western blotting with anti-LBP antibody. Individual relative intensities were plotted, with the value for the standard protein (10 ng) being taken as 100.

FIG. 4. Graphs obtained by quantifying and plotting the intensities of the bands detected by Western blotting with anti-LRG1 [ERR 12362] antibody. Individual relative intensities were plotted, with the value for the standard protein (10 ng) being taken as 100.

FIG. 5. Western blot images obtained with anti-ATG antibody. Samples assigned the same number represent that they are derived from the same patient; the sample from the acute phase and the sample from the recovery phase make a pair.

FIG. 6. Graphs obtained by quantifying and plotting the intensities of the bands detected by Western blotting with anti-ATG antibody. The intensities of the detected bands were directly used as such to prepare the graph.

FIG. 7. Western blot images obtained with anti-LBP, LRG1, AGT and RBP4 antibodies (left panels). Serum samples from 10 Kawasaki disease patients (Nos. 1 to 10) were used (Note: acute phase and recovery phase samples were taken from the same patient). Graphs obtained by quantifying the intensities of the bands detected by Western blotting, with the intensities being directly plotted as such (right panels). NS: non-significant.

FIG. 8. The results of ELISA on LBP, LRG1, AGT and BRP4. Expression levels are compared among the acute and recovery phases of Kawasaki disease and healthy infants (at the time of allergy test). The vertical axis represents the concentration of serum protein. ***: p<0.001, **: p<0.01, *: p<0.1, NS: non-significant

FIG. 9. Changes in LBP, LRG1, AGT and BRP4 based on the results of ELISA. Changes in expression levels among the acute and recovery phases of same 42 KD patients and healthy infants (at the time of allergy test) were examined. The vertical axis represents the concentration of serum protein. A concentration in the acute phase and the corresponding concentration in the recovery phase were connected with a line. ***: p<0.001, **: p<0.01, *p<0.1, NS: non-significant

FIG. 10. The results of ELISA on LBP, LRG1, AGT and BRP4. Expression levels are compared between patients in the acute phase of Kawasaki disease and patients with other pediatric disease. The vertical axis represents the concentration of serum protein. ***: p<0.001, **: p<0.01, *: p<0.1, NS: non-significant

FIG. 11. The results of ROC (receiver operating characteristic curve) analysis of LBP, LRG1, AGT and BRP4. The vertical axis represents sensitivity % (rate at which a person who is truly suffering from KD is tested positive) and the horizontal axis represents 100% -specificity % (rate at which an illness other than Kawasaki disease is misdiagnosed as Kawasaki disease).

FIG. 12. Levels of serum LBP and LRG1 measured on serum samples from patients in the acute phase of Kawasaki disease (55), healthy infants (on allergy test) (13) and patients with pediatric disease (autoimmune disease) (24).

FIG. 13. The results of ROC (Receiver Operating Characteristic curve) analysis. The vertical axis represents sensitivity % (rate at which a person who is truly suffering from Kawasaki disease is tested positive), and the horizontal axis represents 100%—Specificity % (rate at which an illness other than Kawasaki disease is misdiagnosed as Kawasaki disease).

FIG. 14. An embodiment of the test strip for immunochromatography.

FIG. 15. A schematic diagram of the principle of detecting a target molecule (antigen) by immunochromatography.

FIG. 16. Another embodiment of the test strip for immunochromatography.

### EXPLANATION OF SYMBOLS

1 (1a, 1b). Test strip
2. Base sheet

### 3. Membrane

4. Sample pad (sample application site)
5. Conjugate pad (labeling site)
6. Test line (1^{st} detection zone)
7. Control line (2^{nd} detection zone)

### 8. Absorption pad

10. Test strip
5A. Conjugate pad A (1^{st} labeling site for A)
6A. Test line A (1^{st} detection zone for A)
7A. Control line A (2^{nd} detection zone for A)
9A. Capturing member
5B. Conjugate pad B (1^{st} labeling site for B)
6B. Test line B (1^{st} detection zone for B)
7B. Control line B (2^{nd} detection zone for B)

### BEST MODES FOR CARRYING OUT THE INVENTION

Hereinbelow, the present invention will be described in more detail. The present invention provides a method of testing for Kawasaki disease, comprising measuring the level of at least one component selected from the group consisting of lipopolysaccharide binding protein, leucine-rich alpha-2-glycoprotein, angiotensinogen and retinol binding protein 4 in a sample derived from a subject. The above components are useful as Kawasaki disease biomarkers.

As regards the at least one component selected from the group consisting of lipopolysaccharide binding protein, leucine-rich alpha-2-glycoprotein and angiotensinogen, when its level in a sample derived from a subject is high, it may be judged that the subject is likely to be suffering from Kawasaki disease and when its level t is low, it may be judged that the subject is less likely to be suffering from Kawasaki disease. The present inventors have confirmed that these proteins are expressed highly in the acute phase of Kawasaki disease (see Examples described later).

As regards retinol binding protein 4, when its level in a sample derived from a subject is low, it may be judged that the subject is likely to be suffering from Kawasaki disease and when its level is high, it may be judged that the subject is less likely to be suffering from Kawasaki disease. The present inventors have confirmed that the expression of this protein is decreased in the acute phase of Kawasaki disease (see Examples described later).

Therefore, the method of the present invention is applicable to diagnosis of Kawasaki disease (i.e., judgment of whether or not a subject is suffering from Kawasaki disease).

For judging whether or not a subject is suffering from Kawasaki disease, especially making diagnosis in distinction from other diseases, the cutoff values shown under “Acute vs Control” in the lower panel of Table 1 in Example described later may be used. For example, when the serum concentration of lipopolysaccharide binding protein (LBP) is 40.49 μg/ml (concentration with a specificity of 95%) or more, it may be judged that the subject is likely to be suffering from Kawasaki disease; and when the concentration of LBP is less than 40.49 μg/ml, it may be judged that the subject is less likely to be suffering from Kawasaki disease. As regards leucine-rich alpha-2-glycoprotein (LRG1), when the serum concentration is 391.3 μg/ml (concentration with a specificity of 95%) or more, it may be judged that the subject is likely to be suffering from Kawasaki disease; and when the concentration of LRG1 is less than 391.3 μg/ml, it may be judged that the subject is less likely to be suffering from Kawasaki disease. As regards angiotensinogen (AGT), when the serum concentration is 68.83 μg/ml (concentration with a specificity of 95%) or more, it may be judged that the subject is likely to be suffering from Kawasaki disease; and when the concentration of LRG1 is less than 68.83 μg/ml, it may be judged that the subject is less likely to be suffering from Kawasaki disease. As regards retinol binding protein 4 (BRP4), when the serum concentration is 4.575 μg/ml (concentration with a specificity of 95%) or less, it may be judged that the subject is likely to be suffering from Kawasaki disease; and when the serum concentration is more than 4.575 μg/ml, it may be judged that the subject is less likely to be suffering from Kawasaki disease. However, the above-described cutoff values may be altered, by reference to those values shown in the lower panel of Table 1 and based on ROC curves, to another set of criteria, “best” with a specificity of 95%, “better” with a specificity of 90%, and “good” with a specificity of 80%.

Thus, the invention provides method(s) that treat based on measuring that may also include an actual diagnosis based on the measurement of the one or more of lipopolysaccharide binding protein, leucine-rich alpha-2-glycoprotein, angiotensinogen and retinol binding protein 4 as discussed herein.

If a subject is judged to be suffering from Kawasaki disease, treatment of Kawasaki disease should be started. The present invention encompasses a method of treating Kawasaki disease, comprising testing for Kawasaki disease by measuring the level of at least one component selected from the group consisting of lipopolysaccharide binding protein, leucine-rich alpha-2-glycoprotein, angiotensinogen and retinol binding protein 4 in a sample derived from a subject, and providing therapy to the subject.

Kawasaki disease treatment strategy should not be limited to coping with various clinical symptoms observed in the acute phase; it is more important to suppress vasculitis before coronary aneurysm lesions (CALs) appear. Briefly, issues of practical importance include: (1) how to control inflammation by day 7 to 10 of the disease, (2) how to cope with severe cases in which CALs develop by the 7th day of the disease, and (3) how to cope with a case in which inflammation lasts until the 10th day of the disease. The principle of treatment is to suppress early inflammation, and for general cases, (1) intravenous immunoglobulin (IVIG), (2) aspirin anticoagulation therapy, (3) thrombolysis for aneurysm formation and treatments for myocardial infarction and peripheral arterial injury, and (4) treatments for non-cardiovascular complications (meningitis, encephalopathy, DIC, etc.) should be applied.

### I. Standard Therapy

The onset of CALs as a problematic sequela was initially 25 to 30% but decreased to 10-15% since the second half of the 1980s when IVIG was began to be introduced. Long-accumulated data has proven that IVIG therapy is effective in preventing the onset of CALs. In recent years, the Japanese Society of Pediatric Cardiology and Cardiac Surgery has proposed the Clinical Guideline for Medical Treatment of Acute Stage Kawasaki Disease¹⁾. In the guideline, IVIG therapy is a first-line choice. With dose dependency recognized between dose and effect, early diagnosis of therapeutic effects is important, so an IVIG supercritical therapy involving 2 g/kg bolus administration was approved in July 2003 by Japan's Ministry of Health, Labor and Welfare and has been demonstrated for its efficacy and safety as the result of post-use surveillance.. In the United States, too, this therapy has been reported to be effective (https://www.ncbi.nlm.nih.gov/pubmed/14584002 #).

### II. Cures for IVIG Therapy Refractory Cases 1)

In most cases of CALs, IVIG treatment is usually carried out on 3 to 8 days after onset of the disease, so if certain cases turn out to be refractory, the next therapy need be considered after IVIG treatment is completed but then it is often too late from the viewpoint of genesis ofCALs. The frequency of the onset of CALs could be further reduced by identifying IVIG ineffective cases at the early stage and introducing aggressive therapies as shown below.

### 1) Additional Administration of IVIG

If the initial treatment does not improve the symptoms, IVIG is often administered additionally in clinical settings. As described above, this involves excessive injection of a large molecule protein, so the viscosity of blood is elevated to promote undesired formation of thrombus.
2) Methylprednisolone (mPSL) Pulse Therapy
The U.S. Boston group has reported that mPSL pulse therapy is effective for refractory cases, and in Japan, too, reports are being published that show the effectiveness of this therapy. However, in principle, the administration is limited to the early stage of disease (before day 10 of the disease) because it has been suggested that the steroid drug may trigger a delay of the repair mechanism and an increase in thrombogenicity.

### 3) Infliximab

In December 2015, infliximab, a monoclonal antibody against tissue necrosis factor (TNF)-α, was approved as an additional indication for IVIG refractory cases in Japan because it gave good results in clinical trials. However, many issues need be addressed in the future, such as the administration criteria and the method of coping with refractory cases.

### 4) Plasma Exchange Therapy

Since high cytokinemia underlies Kawasaki disease vasculitis, removal of cytokines by plasma exchange therapy (PE) is useful for mitigating inflammation. Putting aside the problems with facilities and equipment, PE has achieved good results even in reviews of severe IVIG refractory cases. With remarkable technological advances, the extracorporeal circulation volume can be reduced to 60-90 ml, and making this therapy applicable to infants weighing 5 kg. Since April 2012, PE has been insurance-covered for Kawasaki Disease and it is now possible to count up to six applications of PE in a series of treatments if the conventional treatment is ineffective.

For details of the above therapies, see the Clinical Guideline for Medical Treatment of Acute Stage Kawasaki Disease (revised in 2012) at http://jspccs.jp/wp-content/uploads/kawasakiguideline2012.pdf

Regarding the administration of warfarin, the maintenance dose starts from between 0.05 to 0.12 mg/kg/day/day 1 and is allowed to reach an optimum range in 4 to 5 days. Prothrombin time (PT) is an item of a screening test for II, V, VII, X coagulation factors and is useful for monitoring the anticoagulant action of warfarin. International standard ratio (PT/INR) is currently used. In KD, the dose is adjusted so that the PT-INR is 1.6 to 2.5 (thrombotest: 10 to 25%). According to the AHA guideline, it is recommended that PT/INR for 0.05 to 0.34 mg/kg be adjusted toward between 2.0 to 2.5 (Circulation 2004; 110 (17): 2747-2771).

For a non-cardiovascular complication (meningitis), a treatment method conforming to the guideline for aseptic meningitis can be performed ((http://wwvv.mhlw.go.jp/stf/shingi/2r98520000013qef-att/2r98520000013r5u.pdf).

For another non-cardiovascular complication (encephalopathy), a treatment method conforming to the pediatric acute encephalopathy clinical practice guideline can be performed (http://minds4.jcqhc.or.jp/minds/child-acute-encephalopathy/child-acute-Encephalopathy.pdf).

For yet another non-cardiovascular complication (DIC), a treatment method conforming to the Japanese version of sepsis clinical practice guideline 2016 CQ16 can be performed (https://www.jstage.jst.go.jp/article/jsicm/24/Supplement2/24_24S0019/_pdf).

Further, if the subject is a patient undergoing treatment of Kawasaki disease, the level of at least one component selected from the group consisting of lipopolysaccharide binding protein, leucine-rich alpha-2-glycoprotein and angiotensinogen in a sample derived from the subject is measured once or more than once at different points of time. When the level is low or decreased, it may be judged that the subject has been recovered from Kawasaki disease by the treatment; and when the level is high or not decreased, it may be judged that the subject has not been recovered or not sufficiently recovered from Kawasaki disease by the treatment. The present inventors have compared patients in the acute and recovery phases of Kawasaki disease and confirmed that the levels of these proteins decreased as the patients recovered from the disease (see the Example described later).

As regards retinol binding protein 4, its level in a sample derived from the subject is measured once or more than once at different points of time. When the level of interest is high or increased, it may be judged that the subject has been recovered from Kawasaki disease by the treatment; and when the level of interest is low or not increased, it may be judged that the subject has not been recovered or not sufficiently recovered from Kawasaki disease by the treatment. The present inventors have compared patients in the acute and recovery phases of Kawasaki disease and confirmed that the level of this protein increased as the patients recovered from the disease (see the Example described later).

Accordingly, the method of the present invention can also be used to confirm changes in the conditions of Kawasaki disease patients, their current conditions, test for prognosis and therapeutic effects on Kawasaki disease

To make judgment about recovery from Kawasaki disease, the cutoff values shown under “Acute vs Recovery” in the lower part of Table 1 in Example described later may be used. For example, if the subject is a patient undergoing treatment of Kawasaki disease, the serum concentration of lipopolysaccharide binding protein (LBP) in a sample derived from the subject is measured once or more than once at different points of time. When the serum concentration is 56.54 μg/ml (concentration with a specificity of 95%) or less, it may be judged that the subject has been recovered from Kawasaki disease by the treatment; and when the serum concentration is more than 56.54 μg/ml, it may be judged that the subject has not been recovered or not sufficiently recovered from Kawasaki disease by the treatment. As regards leucine-rich alpha-2-glycoprotein (LRG1), its serum concentration in a sample derived from the subject is measured once or more than once at different points of v. When the serum concentration is 369.7 μg/ml (concentration with a specificity of 95%) or less, it may be judged that the subject has been recovered from Kawasaki disease by the treatment; and when the serum concentration is more than 369.7 μg/ml, it may be judged that the subject has not been recovered or not sufficiently recovered from Kawasaki disease by the treatment. As regards angiotensinogen (AGT), its serum concentration in a sample derived from the subject is measured once or more than once at different points of time. When the serum concentration is 101.9 μg/ml (concentration with a specificity of 95%) or less, it may be judged that the subject has been recovered from Kawasaki disease by the treatment; and when the serum concentration is more than 101.9 μg/ml, it may be judged that the subject has not been recovered or not sufficiently recovered from Kawasaki disease by the treatment. As regards retinol binding protein 4 (RBP4), its serum concentration in a sample derived from the subject is measured once or more than once at different points of time. When the serum concentration is 6.759 μg/ml (concentration with a specificity of 95%) or more, it may be judged that the subject has been recovered from Kawasaki disease by the treatment; and when the serum concentration is less than 6.759 μg/ml, it may be judged that the subject has not been recovered or not sufficiently recovered from Kawasaki disease by the treatment. However, the above-described cutoff values may be altered, by reference to those values shown in the lower panel of Table 1 and based on ROC curves, to another set of criteria, “best” with a specificity of 95%, “better” with a specificity of 90%, and “good” with a specificity of 80%.

The sample derived from subjects may be exemplified by serum, blood (whole blood), plasma, and so on.

The present invention also provides a test kit for Kawasaki disease, comprising at least one reagent selected from the group consisting of reagents capable of specifically detecting lipopolysaccharide binding protein, reagents capable of specifically detecting leucine-rich alpha-2-glycoprotein, reagents capable of specifically detecting angiotensinogen, and reagents capable of specifically detecting retinol binding protein 4.

As these reagents, antibodies are preferable. Antibodies specifically binding to lipopolysaccharide binding protein, antibodies specifically binding to leucine-rich alpha-2-glycoprotein, antibodies specifically binding to angiotensinogen, and antibodies specifically binding to retinol binding protein 4 may be used. Such antibodies are commercially available. Antibodies may be either monoclonal antibodies or polyclonal antibodies. Antibodies may be labeled with radioisotopes, enzymes, luminescent substances, fluorescent substances, biotin and so on. In the case where the target molecule (in the present invention, LBP, LRG1, ATG or RBP4) is first reacted with a primary antibody which specifically binds it and then this primary antibody is reacted with a secondary antibody which binds it to thereby detect the target molecule, the secondary antibody may be labeled (the primary antibody is not labeled).

In addition to the reagent, the kit of the present invention may further comprise standard proteins (LBP, LRG1, ATG and RBP4), buffers, substrates (when antibodies are labeled with enzymes), reaction stoppers, washing solutions, reaction vessels, instructions for use, and so on.

Further, the present invention provides a method of testing for Kawasaki disease, comprising measuring the levels of lipopolysaccharide binding protein (LBP) and leucine-rich alpha-2-glycoprotein (LRG1) in a sample derived from a subject.

When the LBP level and the LRG1 level are respectively higher than specified values, it is possible to judge that the subject is likely to be suffering from Kawasaki disease; and when the LBP level and the LRG1 level are respectively lower than the specified values, it is possible to judge that the subject is less likely to be suffering from Kawasaki disease.

To know any involvement with Kawasaki disease, especially to diagnose it in distinction from other diseases, specified values (cutoff values) may be used as an index for judgment. For example, in the acute phase of Kawasaki disease, an LBP level of 25 ng/ml or more and an LRG1 level of 300 ng/ml or more will allow for diagnosis of Kawasaki disease with high probability. Therefore, the method of the present invention can be applied to diagnosis of Kawasaki disease (to know any involvement with Kawasaki disease).

Since one threshold is set for each of LBP and LRG1 in the above explanation, binarization is possible for LBP (not less than OR less than) and for LRG1 (not less than OR less than). For example, by assigning “1” or “0” to each of the binary values of LBP and LRG1, measured values may be subjected to digital judgement by software.

To set thresholds, measured values may be analyzed with ROC (Receiver Operating Characteristic curve), followed by setting thresholds depending on their specificity (see Table 2 provided later).

Samples derived from subjects may be liquid clinical samples such as serum, blood (whole blood) or plasma.

Measurement of LBP and LRG1 levels in samples may be performed by any method such as enzyme-linked immunosorbent assay (ELISA), immunoblotting, fluorescent antibody technique (FA), radioimmunoassay (RIA), fluorescent enzyme immunoassay (FLEIA), chemiluminescent enzyme immunoassay (CLEIA), chemiluminescent immunoassay (CLIA), electro-chemiluminescence immunoassay (ECLIA), immunochromatographic assay (ICA), Western blotting (WB) or the like. Preferably, immunochromatography is used because this method enables on-site diagnosis even in small-sized medical facilities that are not provided with special equipment and laboratory technicians.

Further, the present invention provides a test strip for detecting Kawasaki disease by immunochromatography, comprising an anti-LBP antibody immobilized carrier and an anti-LRG1 antibody immobilized carrier.

Antibodies specifically binding to LBP and antibodies specifically binding to LRG1 are commercially available. These antibodies may be either monoclonal or polyclonal antibodies. When immunochromatography is used for testing, antibodies (labeled antibodies) specifically binding to target molecules (LBP and LRG1 in the present invention) may be reacted with the target molecules, followed by reaction with antibodies (unlabeled antibodies) specifically binding to the target molecules for detection of the target molecules. Specific examples of substances for labeling antibodies include, but are not limited to, colored insoluble particles such as colloidal particles (e.g., colloidal metal particles of gold, silver, platinum, etc.) and polystyrene particles colored with red or blue pigments or dyes.

The carrier may be any carrier as long as it is capable of chromatographically developing a substance to be detected (i.e., target molecule) and is capable of immobilizing an antibody specifically binding to the substance to be detected. The carrier is composed of a highly water-absorbent material (such as porous material) so that it is capable of moving by capillary action. Specifically, nylon, polysulfone, polyethersulfone, polyvinyl alcohol, polyester, polyolefin, cellulose, nitrocellulose, cellulose acetate, acetyl cellulose, glass fiber, mixtures thereof, and the like may be enumerated. In particular, nitrocellulose may preferably be used.

An anti-LBP antibody immobilized carrier and an anti-LRG1 antibody immobilized carrier may be included in one test strip. Alternatively, an anti-LBP antibody immobilized carrier and an anti-LRG1 antibody immobilized carrier may be included in different test strips.

The test strip may, for example, be composed of the following components:

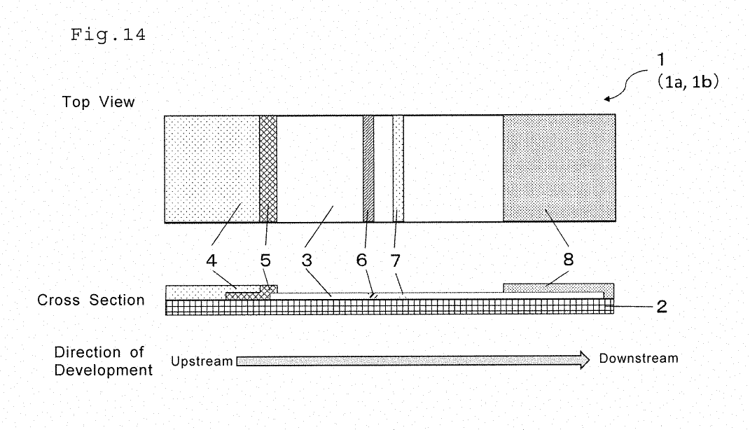

Sample pad for sample application (4 in FIG. 14)

Conjugate pad in which a labeled antibody specifically binding to a target molecule (LBP or LRG1 in the present invention) is immobilized (5 in FIG. 14)

Membrane wherein an unlabeled antibody specifically binding to the target molecule is immobilized on test line (6 in FIG. 14) and an unlabeled antibody specifically binding to the above-mentioned labeled antibody is immobilized on control line (7 in FIG. 14)

Absorption pad (8 in FIG. 14)

Base sheet (2 in FIG. 14)

Sample pad (2 in FIG. 14) is a site onto which a sample liquid is dropped and it may be made from a highly water-absorbent material such as sponge, glass fiber, nylon, cellulose, polyurethane, polyacetate, cellulose acetate or nonwoven fabrics thereof. For removing any solid particles that are unnecessary for the test, the mesh size of the nonwoven fabric may be so selected that the fabric will function as a filter for the sample liquid. The thickness of sample pad 4 is not particularly limited. Preferably, the thickness is 0.1 to 3 mm.

Conjugate pad (5 in FIG. 14) contains a labeled antibody specifically binding to a target molecule (LBP or LRG1 in the present invention).

A water-absorbent material for immobilizing the labeled antibody is used for conjugate pad (labeling site) 5. For example, a nonwoven fabric of sponge, glass fiber or the like may be used. Although the thickness of this pad is not particularly limited, a thickness of 0.1 to 3 mm is preferably used. It is necessary to locate conjugate pad (labeling site) 5 downstream of sample pad 4, upstream of test line 6, and upstream of control line 7.

For preparing the conjugate pad, a labeling substance may be conditioned to have an optimum concentration for detecting a target molecule (LBP or LRG1 in the present invention) and added to an antibody specifically binding to the target molecule to thereby label the antibody. Then, a pad (e.g., a nonwoven fabric of glass fiber) may be impregnated with the resultant labeled antibody, followed by sufficient drying.

As described above, membrane 3 is composed of a porous material. Specifically, nylon, polysulfone, polyethersulfone, polyvinyl alcohol, polyester, polyolefin, cellulose, nitrocellulose, cellulose acetate, acetyl cellulose, glass fiber, mixtures thereof, and the like may be enumerated. In particular, nitrocellulose may preferably be used. Although the thickness of this membrane is not particularly limited, a thickness of 0.1 to 3 mm is preferably used.

At the position of test line (6 in FIG. 14) in membrane 3, an antibody (unlabeled) specifically binding to the target molecule is immobilized. The shape of the test line is shown to be linear. Alternatively, a plurality of dots may be aligned in a linear shape. Further, the line is not limited to a straight line and may be a circular arc or a curve.

On control line (7 in FIG. 14) of membrane 3, an antibody (unlabeled) specifically binding to the above-described labeled antibody is immobilized. Specific examples of antibodies to be immobilized on the control line include, but are not limited to, anti-mouse IgG antibody, anti-goat IgG antibody, anti-rabbit IgG antibody and anti-rat IgG antibody. The shape of the control line is shown to be linear. Alternatively, a plurality of dots may be aligned ina linear shape. Further, the line is not limited to a straight line and may be a circular arc or a curve. Absorption pad 8 which absorbs the sample liquid and labeled antibody that have flown thereto by capillary action is capable of controlling the direction of the liquid flow.

Absorption pad 8 is positioned on test strip 1 at an end different from the end where sample pad 4 is positioned. By positively absorbing the sample liquid moving on test strip 1, absorption pad 8 generates a uniform flow in the sample liquid, eventually forming an upstream and a downstream (sample pad 4 is on an upstream side and absorption pad 8 a downstream side). Absorption pad is composed of a water-absorbent material so that it can absorb a large volume of liquid. For example, a nonwoven fabric of cellulose, cellulose acetate, glass fiber, etc. may be used as such material. Although the thickness of this pad is not particularly limited, a thickness of 0.1 to 3 mm is preferably used.

Base sheet 2 is a backing for membrane 3, sample pad (sample application site) 4, conjugate pad (labeling site) 5 and absorption pad 8, and is composed of a liquid-impenneable material such as synthetic resin. As another function, base sheet 2 keeps membrane 3 and other components integral as to retain a certain degree of strength and prevents the sample liquid from flowing out of test strip 1. As the synthetic resin, polyethylene terephthalate (PET) may be used. Although the thickness of this base sheet is not particularly limited, a thickness of 0.1 to 3 mm is preferably used. Base sheet 2 is in close contact with membrane 3, sample pad (sample application site) 4, conjugate pad (labeling site) 5 and absorption pad 8 to constitute test strip 1. In order to prevent detachment during transportation, an adhesion layer consisting of an adhesive may be provided at the interface between base sheet 2 and other components (e.g., interface between base sheet 2 and absorption pad 4; interface between base sheet 2 and conjugate pad 5; interface between base sheet 2 and membrane 3; and interface between base sheet 2 and absorption pad 8).

Although the size of test strip 1 is not particularly limited, a size of 0.5-20 mm in width and 10-100 mm in length is preferable because it is easy to handle and enables easy judgment as by visual observation during diagnosis. Besides, the sample liquid need be used in a smaller amount. Individual components are cut to a size which is the same as or smaller than that of test strip 1; the thus cut components are then assembled together.

Test strip 1 may immediately be used as a dip stick type strip. Alternatively, test strip 1 may be used as a test stick contained in a plastic case provided with openings for a sample application site and a judgement site.

One example of preparation of test strip 1 of the present invention for detecting Kawasaki disease by immunochromatography will be described below with reference to FIG. 14. An antibody (labeled antibody) linked to a labeling substance specifically binding to an antigen to be detected is applied to conjugate pad (labeling site) 5. On test line (1^{st} detection zone) 6 of membrane 3 backed with base sheet 2, a solid phase is formed from an antibody capable of specifically binding to an antigen to be detected (LBP or LRG1 in the present invention). On control line (2^{nd} detection zone) 7, a solid phase is foliated from a substance specifically binding to the labeled antibody (labeled antibody specific antibody). Conjugate pad (labeling site) 5 and membrane 3 are laminated with other components (sample pad 4 and absorption pad 8) and cut to an appropriate width, whereby test strip 1 for immunochromatography is prepared.

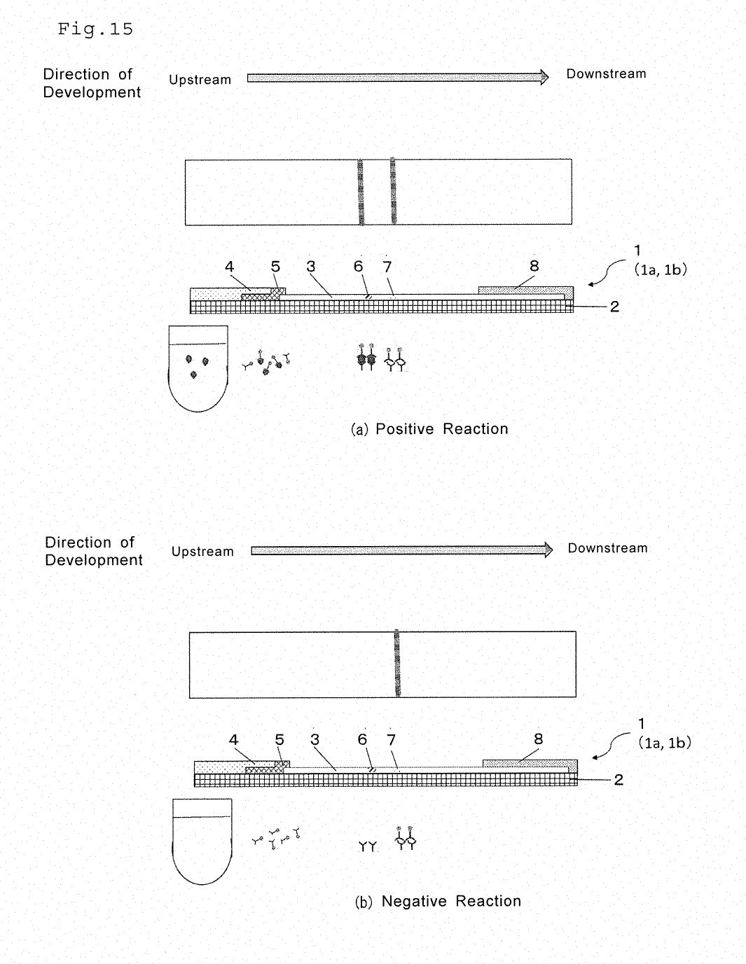

A method of using the above-described test strip 1 (the principle of immunochromatography) will be described below with reference to FIG. 15. When a sample liquid is dropped onto sample application site 4 of test strip 1, the sample liquid containing the antigen bound to the labeled antibody flows downstream on membrane 3 by capillary action to be absorbed in absorption pad 8. As a result, the solid phase of antibody, the substance to be detected (antigen), and the labeled antibody form a complex in 1^{st} detection zone 6 on test strip 1 to become visible. The labeled antibody which has not been captured in 1^{st} detection zone 6 continues to flow until it reaches 2^{nd} detection zone 7 located downstream of 1^{st} detection zone 6, where the substance binding to the labeled antibody and the labeled antibody form a visible complex. By these results of visualization, it is shown that an antigen-antibody reaction has progressed normally. Therefore, if the sample is positive, two lines, i.e., test line and control line, are observed (FIG. 15, (a) positive reaction); and if the sample is negative, control line alone is observed (FIG. 15, (b) negative reaction).

The signal emitted by labeled antibodies may be observed visually or measured with a detection device suitable for the labeling substance. If the labeling substance is colored insoluble carrier particles such as colloidal particles or polystyrene particles, a densitometer may be used as the detection device; if the labeling substance is a fluorescent dye, a fluorescence detector may be used. Alternatively, the so-called immunochromatography reader may also be used.

Test strip 1 may be packaged together with an instruction manual to thereby prepare a test kit for Kawasaki disease.

The instruction manual may include such information as testing procedures, judging method, precautions for use and handling, as well as the storage conditions and expiration date of test strip 1.

### EXAMPLES

Hereinbelow, the present invention will be described in detail with reference to the following Examples.

### Example 1

### (Methods)

Serum samples from 55 Kawasaki disease (KD) patients in the acute phase and 51 KD patients in the recovery phase were used (those samples were supplied by Yokohama City University Hospital; Yokohama City University Medical Center; Kanagawa Children's Medical Center; and Showa General Hospital). The operations described below were performed on two types of proteins.

### Confirmation of the Expression Level of Each Protein in Serum by Western Blotting

An aliquot of 0.1 μl from each serum was mixed with a sample buffer. After heating at 95° C. for 5 minutes, the resultant solution was centrifuged at 15,000 rpm at room temperature for 5 minutes to thereby prepare a sample. The sample was electrophoresed at a constant voltage of 300V on Perfect NT Gel (DRC) to isolate proteins. After electrophoresis, the proteins were transferred onto a PVDF membrane from the Perfect NT Gel with a semi-dry blotting device (Trans-Blot Turbo System (BioRad)). The resultant PVDF membrane was soaked in a blocking solution and shaken at room temperature for 1 hour to perform blocking treatment. The thus treated PVDF membrane was reacted with an antibody diluted with antibody dilution buffer (anti-LBP antibody (GeneTex) diluted at 1:3000 or anti-LRG1 [EPR 12362] antibody (Abcam) diluted at 1:5000) at room temperature for 16-18 hours. After the reaction, each PVDF membrane was washed with 0.05% [v/v] Tween 20-containing TBS (TBS-T) for 10 minutes three times, and then reacted with standard anti-rabbit IgG-HRP diluted at 1:5000 with antibody dilution buffer at room temperature for 1 hour. After the reaction, the membrane was washed again with TBS-T for 10 minutes three times. Then, the protein of interest was detected with LAS-4000 EP UV mini PRH (Fujifilm) using ECL Select Western Blotting Detection Reagent (GE Healthcare) as a substrate.

Subsequently, band intensities in the resultant images were quantified with MultiGauge Analysis Software (ver. 3.11, Fujifilm). Using a commercial recombinant protein (Recombinant Human LBP (R&D Systems) [2.5 ng] or Recombinant Human LRG1 (Novoprotein) [10 ng]) as a standard protein for quantitative analysis, relative intensity of each band was calculated taking the band intensity of the standard protein as 100. Graphs were prepared with the resultant numerical figures. Further, Mann-Whitney test was carried out between the acute and the recovery phases using GraphPad Prism (ver. 5, MDF).

### (Results)

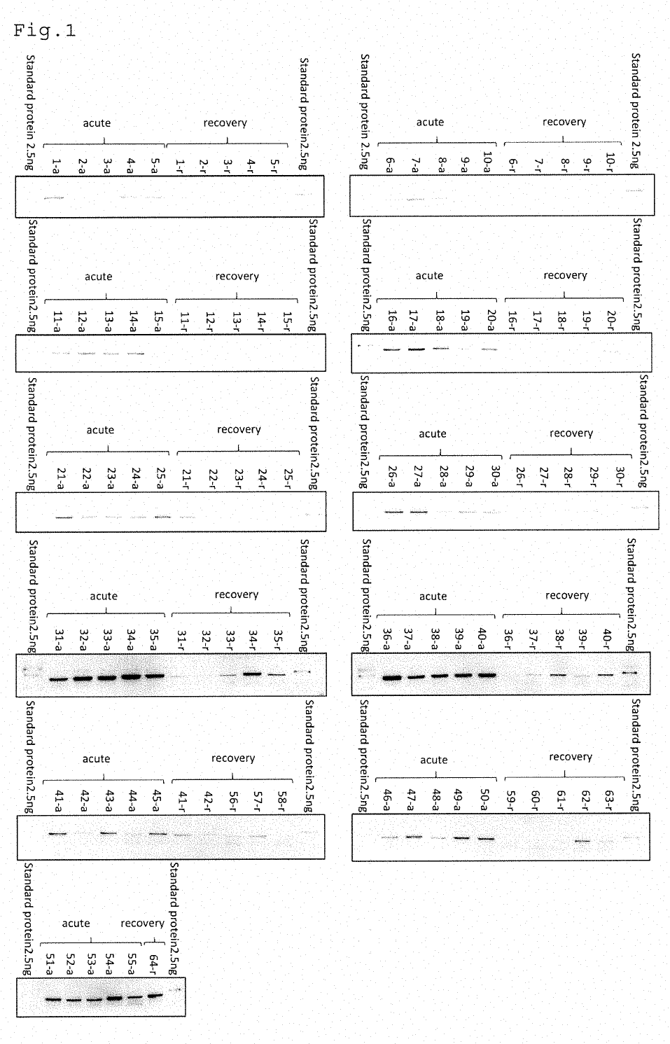

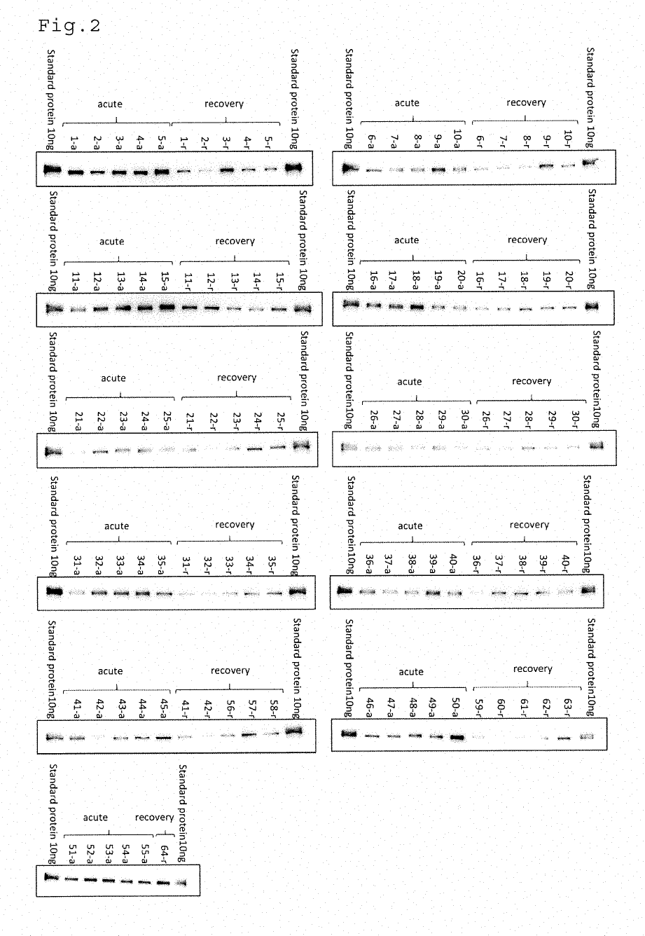

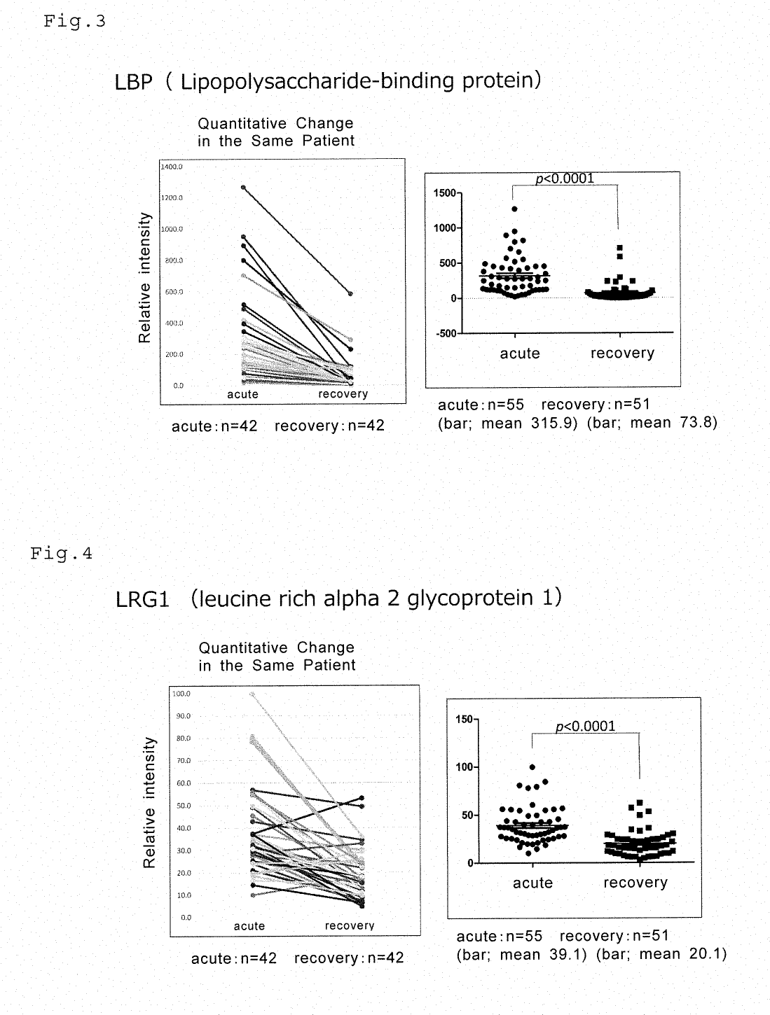

The present inventors validated whether proteins LBP and LRG1 were specifically expressed in the acute phase of Kawasaki disease by Western blot analysis using antibodies to these proteins. Usually, proteins of high concentrations will hinder isolation of other proteins in electrophoresis. However, LBP and LRG1 of interest in the present experiment were expressed at high levels and due to the presence of antibodies that were highly specific for these proteins, the amount of serum used to detect each of these proteins was as small as 0.1 μl. Therefore, in the present experiment, all the serum samples from 55 patients with KD in the acute phase and 51 patients with KD in the recovery phase were subjected to SDS-PAGE gel electrophoresis to examine expression levels without removing proteins of high concentrations (FIGS. 1 and 2). As a result, a significant difference (p<0.0001) was observed between the acute phase and the recovery phase with respect to the expression levels of LBP and LRG1. These results are shown in FIG. 3 for LBP and FIG. 4 for LRG1.

### (Discussion)

LBP is a protein occurring in blood at high concentrations during bacterial infection (International Immunology, 22:271-280, 2010). LBP has high affinity for lipopolysaccharides (LPS), a component constituting the cell membrane of gram negative bacteria, and forms complexes. It is known that these LBP-LPS complexes are delivered to CD14 existing on the cell membrane of macrophages, etc., bind to toll-like receptor 4 (Journal of Periodontal Research, 49:1-9, 2014) and activate signaling pathways to thereby promote secretion of various inflammatory cytokines. Further, it has been reported that LBP expression increases in childhood febrile urinary infection and sepsis (Pediatric Nephrology, 28, 1091-1097, 2013). Therefore, bacterial or otherwise infection might have also occurred in Kawasaki disease to cause an eventual increase in LBP expression.

As for LRG1, it also occurs in blood and has been identified as a novel inflammation marker protein. It has been reported that LRG1 expression is increased by rheumatoid arthritis, cancer, inflammatory bowel diseases, macrophage activation by LPS administration, and so on (Annals of the Rheumatic Diseases, 69:770-774, 2010; Biochem Biophys Res Commun, 382:776-779, 2009; Proc Natl Acad Sci USA. 110,E2332-E2341, 2013). Recently, it has been reported that LRG1 promotes angiogenesis via regulation of signal transduction of transforming growth factor-β (TGF-β) (Nature, 499:306-311, 2013). TGF-β is also involved in the expression of VEGF which has been reported to occur at high concentrations in the serum of Kawasaki disease patients in the acute phase (Pediatric Research, 44:596-599, 1998). There is also a report that inflammation and angiogenesis are observed in coronary artery aneurysms and cardiac muscles of Kawasaki disease patients (Pediatric Cardiology, 26:578-584, 2005). Therefore, it is suggested that LRG1 may be involved in the formation of coronary artery aneurysms in cardiac muscles or in the occurrence of inflammations in cardiac muscles.

Further, LBP and LRG1 have high concentrations in blood and can be detected easily. Therefore, if a diagnostic method using the expression levels of both proteins as diagnostic criteria is employed in addition to the conventional diagnostic method based on major symptoms which is often affected by the subjectivity or experience of physicians to involve a risk of misdiagnosis or oversight, there would be a possibility for accurate and quick diagnosis.

### Example 2

### (Methods)

Serum samples from 20 Kawasaki disease (KD) patients in the acute phase and 20 KD patients in the recovery phase were used (those samples were supplied by Yokohama City University Hospital; Yokohama City University Medical Center; Kanagawa Children's Medical Center; and Showa General Hospital). The operations described below were performed on two types of proteins.

### Confirmation of the Expression Level of Protein in Serum by Western Blotting

An aliquot of 0.05 μl from each serum was mixed with a sample buffer. After heating at 95° C. for 5 minutes, the resultant solution was centrifuged at 15,000 rpm at room temperature for 5 minutes to thereby prepare a sample. The sample was electrophoresed at a constant voltage of 300V on Perfect NT Gel to isolate proteins. After electrophoresis, the proteins were transferred onto a PVDF membrane from the Perfect NT Gel with a semi-dry blotting device (Trans-Blot Turbo System). The resultant PVDF membrane was soaked in a blocking solution and shaken at room temperature for 1 hour to perform blocking treatment. The thus treated PVDF membrane was reacted with anti-AGT antibody (IBL) diluted at 1:100 with antibody dilution buffer at room temperature for 16-18 hours. After the reaction, the PVDF membrane was washed with TBS-T for 10 minutes three times, and then reacted with standard anti-mouse IgG-HRP diluted at 1:5000 with antibody dilution buffer at room temperature for 1 hour. After the reaction, the membrane was washed again with TBS-T for 10 minutes three times. Then, the protein of interest was detected with LAS-4000 EP UV mini PRH using ECL Select Western Blotting Detection Reagent as a substrate. Subsequently, band intensities in the resultant images were quantified with MultiGauge Analysis Software. Graphs were prepared with the resultant numerical figures. Further, Mann-Whitney test was carried out between the acute and the recovery phases using GraphPad Prism (ver. 5, MDF).

### (Results)

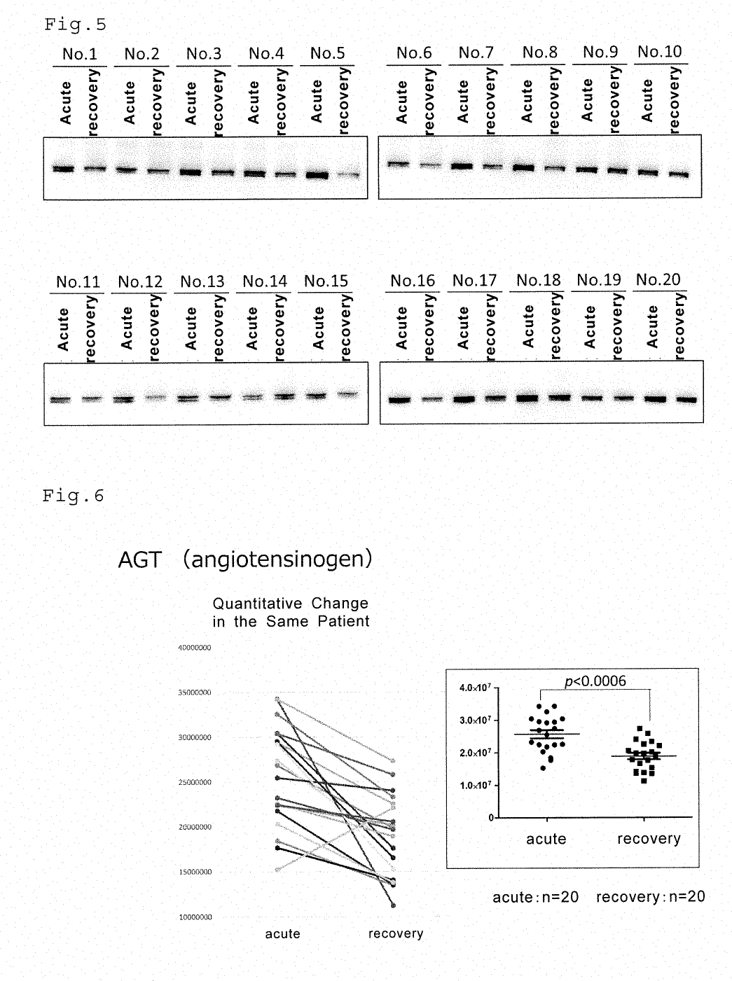

The present inventors validated whether AGT was specifically expressed in the acute phase of Kawasaki disease by Western blot analysis using an antibody to this protein. Like LBP and LRG1, AGT was expressed at high levels and there was an antibody highly specific for this protein. Thus, the amount of serum used to detect the protein was as small as 0.05 μl. For this reason, in the present experiment, all the serum samples from 20 KD patients in the acute phase and 20 KD patients in the recovery phase were subjected to SDS-PAGE gel electrophoresis to examine expression levels without removing proteins of high concentrations (FIG. 5). As a result, a significant difference (p<0.0006) was observed between the acute phase and the recovery phase with respect to the expression level of AGT (FIG. 6).

### (Discussion)

AGT is a precursor of angiotensin, and degraded into angiotensin I and II in the renin-angiotensin system. AGT increases in hypertension, diabetes and chronic nephritis and is believed to play an important role in the onset and progress of hypertension and renal dysfunction. However, the relation between AGT and Kawasaki disease has not been known so far and is an observation that has been first obtained in the present invention.

### Example 3

### (Methods)

Serum samples were supplied by Yokohama City University Hospital, Kanagawa Children's Medical Center, Showa General Hospital, National Institute of Infectious Diseases, Kobe University Hospital, Japanese Red Cross Wakayama Medical Center and Yokohama City University Medical Center (Table 1, upper panel). General consent was obtained from all the patients/subjects who supplied the samples.

KD patients' acute phase serum (Acute): Serum from 55 patients in the febrile period before intervention

KD patients' recovery phase serum (Recovery): Serum from 51 patients in the afebrile period following intervention

Viral infection patients' serum (G1): 106 patients

(RS virus: 21 patients; influenza A virus: 23 patients; influenza B virus: 20 patients; rotavirus: 20 patients; norovirus: 7 patients; adenovirus: 3 patients; and pharyngeal adenovirus: 12 patients)

Bacterial infection patients' serum (G2): 21 patients

(Streptococcus pneumoniae: 1 patient; Klebsiella pneumoniae: 1 patient; gram negative Bacillus: 1 patient; gram negative Bacillus: 1 patient; hemolytic streptococcus: 7 patients; Escherichia coli: 3 patients; Staphylococcus aureus: 2 patients; Staphylococcus epidennidis: 1 patient; Micrococcus: 1 patient; Serratia: 1 patient; and Clostridium difficile: 1 patient)

Autoimmune patients' serum (G3): 24 patients

(idiopathic thrombocytopenic purpura: 3 patients; pediatric rheumatism: 2 patients; GVHD (graft-vs-host disease): 1 patient; VAHS (virus-associated hemophagocytic syndrome): 1 patient; and juvenile idiopathic arthritis: 17 patients)

Healthy subjects' serum (collected at allergy tests) (Healthy): 13 subjects

### Confirmation of Expression Levels of Individual Proteins in Serum by Western Blotting

Serum samples were obtained from 10 Kawasaki disease patients (Note: acute phase and recovery phase samples were taken from the same patient). These samples were diluted with PBS-T. The diluted serum was mixed with 2× sample buffer in equal amounts, and the resultant solution was mixed with Milli-Q water to give a total volume of 10 μl, so that 0.1 μl of serum would be contained per well. Samples for quantitative analysis of LRG1 were heated at 95° C. for 5 minutes before use. Subsequently, supernatants obtained by centrifuging the samples at 21,600× g for 5 minutes at room temperature were used as samples for electrophoresis. A prepared gel was placed in an electrophoresis bath which was then filled with an electrode liquid. The sample was poured into each well, followed by electrophoresis at a constant voltage of 300V. Thus, serum proteins were isolated.

After the electrophoresis, the proteins were transferred onto a PVDF membrane using a transfer device. After the transfer, the PVDF membrane was soaked in a blocking solution and shaken at room temperature for 1 hour to perform blocking treatment. The thus treated PVDF membrane was reacted for 16-18 hours with primary antibodies diluted with antibody dilution buffer (individual antibodies were diluted at the following ratios: anti-LRG1 1/5000; anti-AGT antibody 1/100; anti-BRP4 antibody 1/1000). After the reaction, each PVDF membrane was washed with TBS-T for 10 minutes three times, and then reacted with standard anti-rabbit IgG-HRP or anti-mouse IgG—diluted at 1:5000 with antibody dilution buffer—at room temperature for 1 hour. After the reaction, the membrane was washed again with TBS-T for 10 minutes three times. Then, using a secondary labeled antibody detection reagent as a substrate, the protein of interest was photographed with LAS-4000 EP UV mini PRI-I. Band intensities were quantified with MutiGauge Analysis Software.

### Validation of Kawasaki Disease Specificity by ELISA

Serum concentrations of Kawasaki disease-related proteins LBP, LRG1, AGT and RBP4 in Kawasaki disease (KD) patients, patients with pediatric disease other than KD and healthy subjects were measured by ELISA, and significance test was performed among the above groups. KD patients' serum samples (55 samples from the acute phase and 51 samples from the recovery phase) were used together with samples from healthy infants (13 samples) and patients with other pediatric diseases (106 samples with viral infection, 21 samples with bacterial infection and 24 samples with autoimmune disease) as control groups. For LBP, serum samples were diluted at 1/4000; for LRG1, serum samples were diluted at 1/5000; for AGT, serum samples were diluted at 1/10000; and for RBP4, serum samples were diluted at 1/2500. Reagents such as dilution solution, washing solution or detection reagent, and methods such as reaction time were in accordance with the protocol attached to the ELISA kit for each protein.

### Statistical Analysis

Significance test between KD acute phase and recovery phase with respect to the expression levels in patients' serum as obtained from the results of Western blotting and another significance test between KD acute phase and other groups (KD recovery phase, healthy infants and patients with other pediatric diseases) on the serum concentrations of individual proteins as obtained from the results of ELISA were performed with the statistical analysis software Graph Pad Prism. Further, for validation of utility as a Kawasaki disease biomarker, ROC analysis was performed between KD acute phase (55 samples) and recovery phase (51 samples) and between KD acute phase and other pediatric diseases (144 samples as described above) to calculate AUC.

### (Results)

### Detection of Novel Kawasaki Disease-Related Protein Candidates by Western Blotting

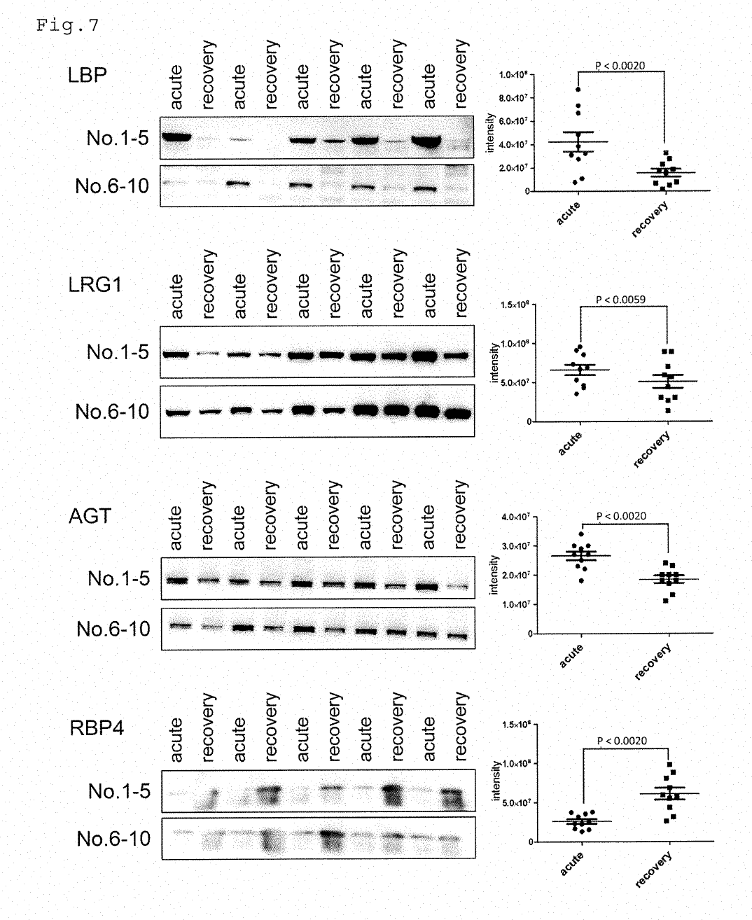

From the Kawasaki disease-related protein candidates obtained from the results of serum proteomic analysis, those proteins which would vary specifically in the level of expression in KD acute phase sera were investigated by Western blotting. As a result, it was newly found that the expression of a particular protein RBP4 in serum significantly decreased in the acute phase (p<0.002) (FIG. 7).

### Validation of Specificity of Kawasaki Disease-Related Proteins by ELISA

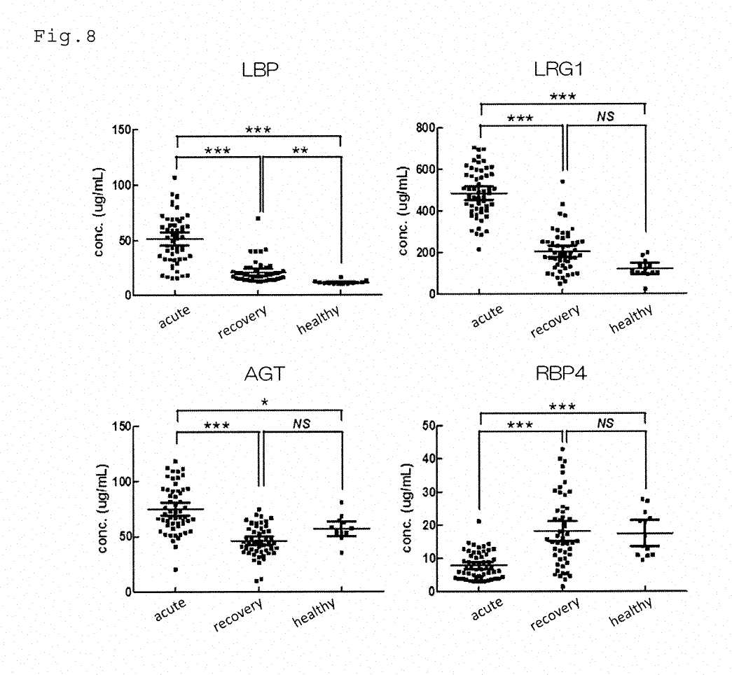

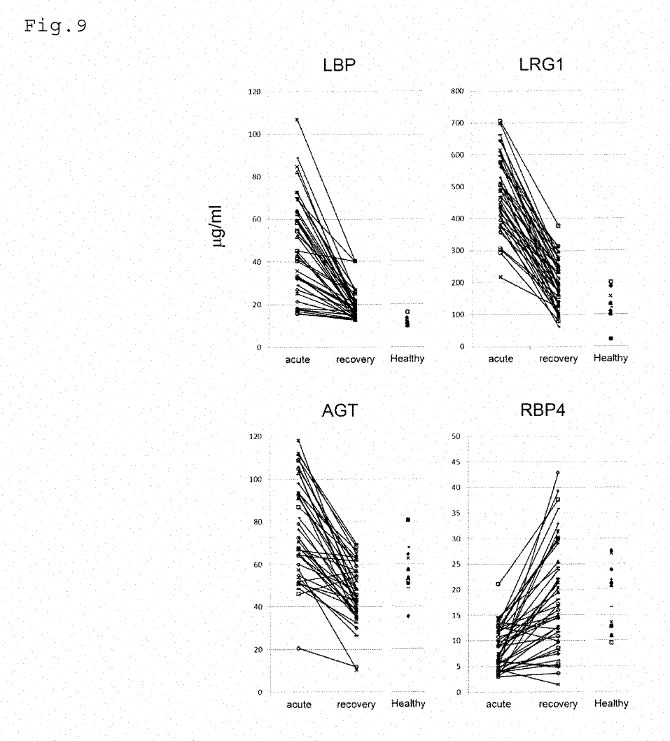

As regards the previously found Kawasaki disease-related proteins LBP, LRG1 and AGT and the newly found RBP4, serum protein concentrations were measured by ELISA using KD patients' serum (acute and recovery phases), healthy infants' serum, and sera from patients with pediatric diseases other than KD (viral infection, bacterial infection and autoimmune disease) (Table 1, upper panel). The results of comparison of individual protein concentrations in sera from KD patients and healthy subjects revealed that expressions of all the four proteins LBP, LRG1, AGT and RBP4 in serum significantly varied between the acute and recovery phases of KD (p<0.001) (FIG. 8). In particular, it was confirmed that the expression of LRG1 decreased in all the patients as they recovered (FIG. 9). Further, as regards LBP, LRG1 and RBP4, a significant difference was also recognized in the comparison between KD acute phase patients and healthy subjects (p<0.001) (FIG. 9).

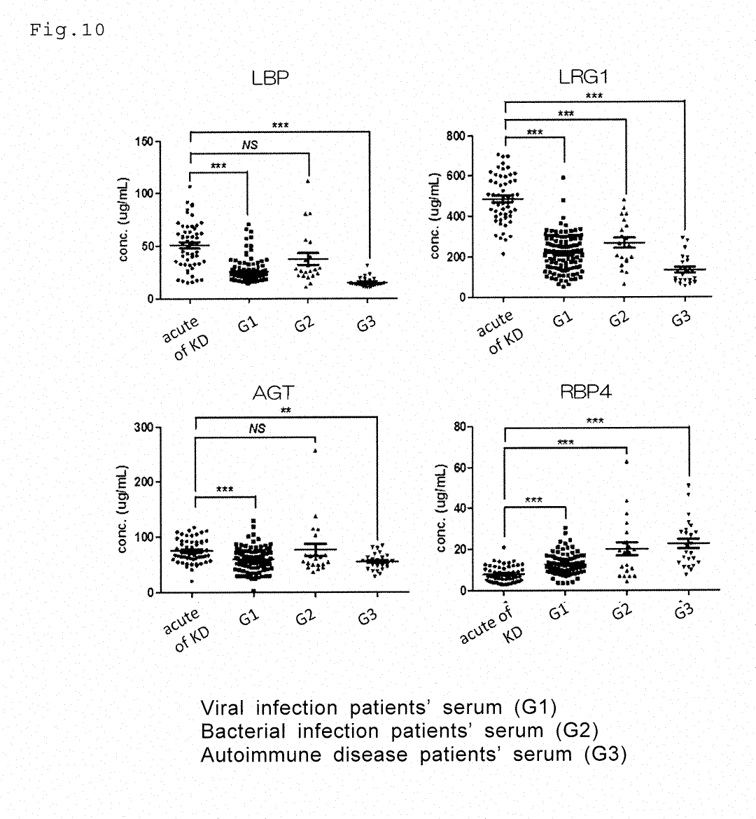

Serum concentrations of LBP, LRG1, AGT and RBP4 in patients with pediatric diseases other than KD were examined by ELISA (FIG. 10; Table 1, upper panel). As a result, a significant difference was recognized in all the four proteins between KD acute phase patients and viral infection or immune disease patients. On the other hand, comparison between KD acute phase patients and bacterial infection patients showed a significant difference in LGR1 and RBP4, but not in LBP and AGT.

### Validation of Biomarker Specificity/Sensitivity by ROC Analysis

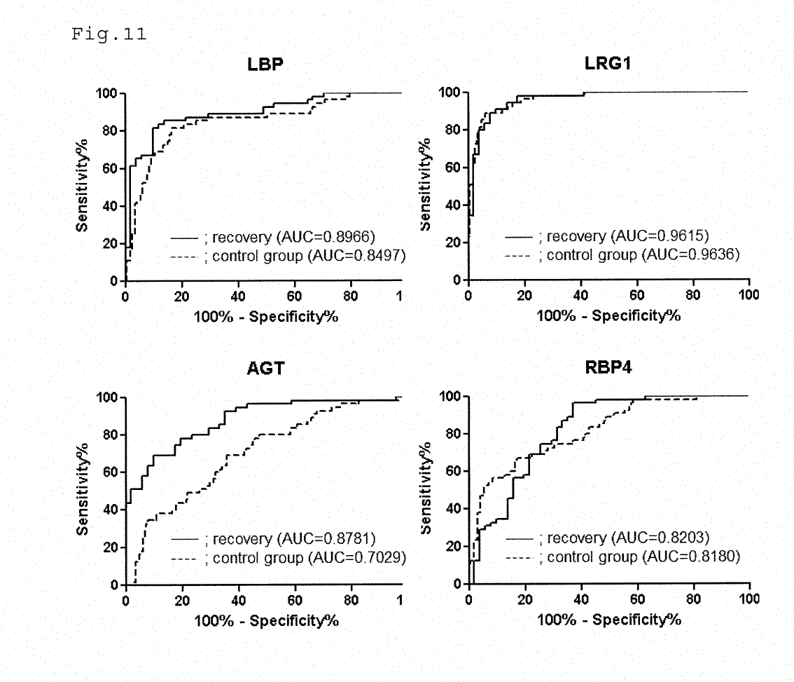

In order to clarify the disease specificity and utility of Kawasaki disease-related proteins LBP, LRG1, AGT and RBP4 in the diagnosis of Kawasaki disease, ROC curves were prepared between KD acute and recovery phases and between KD acute phase and other pediatric diseases (FIG. 11). Diagnostic performance was judged by the magnitude of AUC values. The results revealed that the AUC value of LRG1 was 0.9615 between KD acute phase and recovery phase and 0.9636 between KD acute phase and other diseases, showing that among the four proteins, LRG1 performs bestin the diagnosis of KD and its differentiation from other diseases. Further, the AUC value of LBP was 0.8966 between KD acute phase and recovery phase and 0.8497 between KD acute phase and other diseases, thus showing that next to LRG1, LBP is the most useful in the diagnosis of KD.

Further, cutoff value, sensitivity and specificity were calculated using the statistical analysis software Graph Pad Prism based on the data shown in FIG. 11 (Table 1, lower panel).

Cutoff value (best): the concentration giving a specificity of 95%
Cutoff value (better): the concentration giving a specificity of 90%
Cutoff value (good): the concentration giving a specificity of 80%
Sensitivity: the rate at which a patient who is truly suffering from KD is diagnosed positive 100%—Specificity: the rate at which a patient suffering from an illness other than KD is misdiagnosed as suffering from KD

TABLE 1
Patient information used in ELISA, serum concentrations of LBP, LRG1, AGT and RBP4 in
individual groups as determined by ELISA (mean), and individual cutoff values (best, better and good)

| | | KD | | | | | |
|---|---|---|---|---|---|---|---|
| | | | Recovery | Group 1 | Group 2 | Group 3 | Healthy |
| | | Acute (n = 55) | (n = 51) | (n = 106) | (n = 21) | (n = 24) | (n = 13) |
| Age (range (median)) | | 0-12 (2) | 0-12 (2) | 0-15 (2) | 0-17 (7) | 2-18 (10.5) | 0-10 (4) |
| Male (%) | | 54.5 | 60.8 | 60.4 | 52.4 | 25.0 | 46.2 |
| KD | days | 2-10 | 5-32 | — | — | — | — |
| Conc. | LBP (mean ± SD) | 51.1 ± 22.2 | 21.0 ± 10.1 | 25.7 ± 11.3 | 37.6 ± 25.5 | 15.1 ± 4.9 | 11.6 ± 1.8 |
| (μg/ml)* | LRG1 (mean ± SD) | 487.3 ± 117.1 | 206.4 ± 99.4 | 217.6 ± 93.2 | 268.2 ± 115.1 | 134.1 ± 67.6 | 120.6 ± 44.8 |
| | AGT (mean ± SD) | 74.8 ± 21.3 | 45.9 ± 14.0 | 58.9 ± 21.9 | 76.1 ± 49.5 | 54.9 ± 15.1 | 56.8 ± 10.9 |
| | RBP4 (mean ± SD) | 7.9 ± 4.0 | 18.2 ± 10.6 | 12.7 ± 4.9 | 20.2 ± 14.5 | 22.6 ± 11.5 | 17.5 ± 6.6 |

| Acute vs Control | | | | | Acute vs Recovery | | | | |
|---|---|---|---|---|---|---|---|---|---|
| | LBP | LRG1 | AGT | RBP4 | | LBP | LRG1 | AGT | RBP4 |
| best | | | | | best | | | | |
| Specificity (95% or more) | 95.12 | 95.12 | 95.12 | 95.12 | Specificity (95% or more) | 96.08 | 96.08 | 96.08 | 96.08 |
| Sensitivity % | 43.64 | 85.45 | 16.36 | 47.27 | Sensitivity % | 65.45 | 80.00 | 50.91 | 29.09 |
| Cutoff value (μg/ml) | 56.54 | 369.70 | 101.90 | 6.76 | Cutoff value (μg/ml) | 40.49 | 391.30 | 68.83 | 4.58 |
| better | | | | | better | | | | |
| Specificity (90% or more) | 90.24 | 90.24 | 90.24 | 90.24 | Specificity (90% or more) | 90.20 | 90.20 | 90.20 | 90.20 |
| Sensitivity % | 67.27 | 89.09 | 34.55 | 56.36 | Sensitivity % | 81.82 | 90.91 | 69.09 | 34.55 |
| Cutoff value (μg/ml) | 38.90 | 327.90 | 83.67 | 7.64 | Cutoff value (μg/ml) | 31.00 | 313.80 | 63.72 | 5.26 |
| good | | | | | good | | | | |
| Specificity (80% or more) | 80.49 | 80.49 | 80.49 | 80.49 | Specificity (80% or more) | 80.39 | 80.39 | 80.39 | 80.39 |
| Sensitivity % | 81.82 | 96.36 | 43.64 | 67.27 | Sensitivity % | 85.45 | 98.18 | 78.18 | 58.18 |
| Cutoff value (μg/ml) | 29.66 | 291.70 | 74.42 | 9.26 | Cutoff value (μg/ml) | 25.31 | 275.60 | 59.45 | 8.40 |

*Concentrations of LBP, LRG1, AGT and RBP4 in serum were determined with ELISA.

### (Discussion)

Retinol binding protein (RBP) is a protein with a molecular weight of 21 kDa that is synthesized in the liver and is capable of binding and secreting vitamin A (retinol) accumulated in the liver so that it is transported to target organs (cells). RBP4 is an RBP produced in the liver or adipocytes and is also designated as plasma RBP (PRBP) since it is secreted into blood (plasma). It has been pointed out that RBP4 is involved in diabetes and insulin resistance, and this protein is used as a marker that quickly reflects nutrient conditions and the protein synthesis capacity of the liver. However, the relation of RBP4 with Kawasaki disease is not known.

The etiology of Kawasaki disease is still unknown and it is suggested that some abnormality occurring in the immune system might cause the pathology of KD. Inflammatory proteins such as CRP or SAA are present in excess in the sera of KD patients in acute phase, and the serum concentrations of these proteins are examined in common blood test as reference items. However, many of such inflammatory proteins reflect nonspecific, systemic inflammations and vasculitis and they do not serve to differentiate KD in a specific way.

Therefore, it is important to develop those diagnostic markers other than such proteins which are capable of distinguishing KD from other diseases. According to the present invention, it has been suggested that Kawasaki disease can be specifically diagnosed by examining serum concentrations of Kawasaki disease-related proteins LBP, LRG1, AGT and RBP4 in patients. In particular, LBP and LRG1 have been found to have good diagnostic performance. The pathology of Kawasaki disease covers an extremely wide range and a number of mechanisms are predictably involved in the development of Kawasaki disease. Since all of the four types of proteins discovered in the present invention are found in blood at high concentrations, the present inventors believe that a simple and highly precise diagnostic method for Kawasaki disease can be developed by using these proteins as indicators.

### Example 4

### (Methods)

Serum samples were supplied by Yokohama City University Hospital, Kanagawa Children's Medical Center, Showa General Hospital and Yokohama City University Medical Center. General consent was obtained from all the patients/subjects who supplied the samples.

KD patients' acute phase serum (acute): Serum from 55 patients in the febrile period before intervention

Autoimmune patients' serum (G3): 24 patients

(idiopathic thrombocytopenic purpura: 3 patients; pediatric rheumatism: 2 patients; GVHD (graft-vs-host disease): 1 patient; VAHS (virus-associated hemophagocytic syndrome): 1 patient; and juvenile idiopathic arthritis: 17 patients)

Healthy subjects' serum (collected at allergy tests) (Healthy): 13 subjects

### Measurement of Serum Concentrations of Kawasaki Disease-Related Proteins LBP and LRG1 by ELISA

Serum concentrations of Kawasaki disease-related proteins LBP and LRG1 were measured in Kawasaki disease (KD) patients (55 samples) and control groups consisting of patients with autoimmune disease (24 samples) and healthy subjects (13 samples) by ELISA. For LBP, serum samples were diluted at 1/4000 and for LRG1, serum samples were diluted at 1/5000. Reagents such as dilution solution, washing solution or detection reagent, and methods such as reaction time were in accordance with the protocol attached to the ELISA kit for each protein.

### (Results)

### Validation of the Usefulness of LBP and LRG1 as Biomarkers for Diagnosing Kawasaki Disease

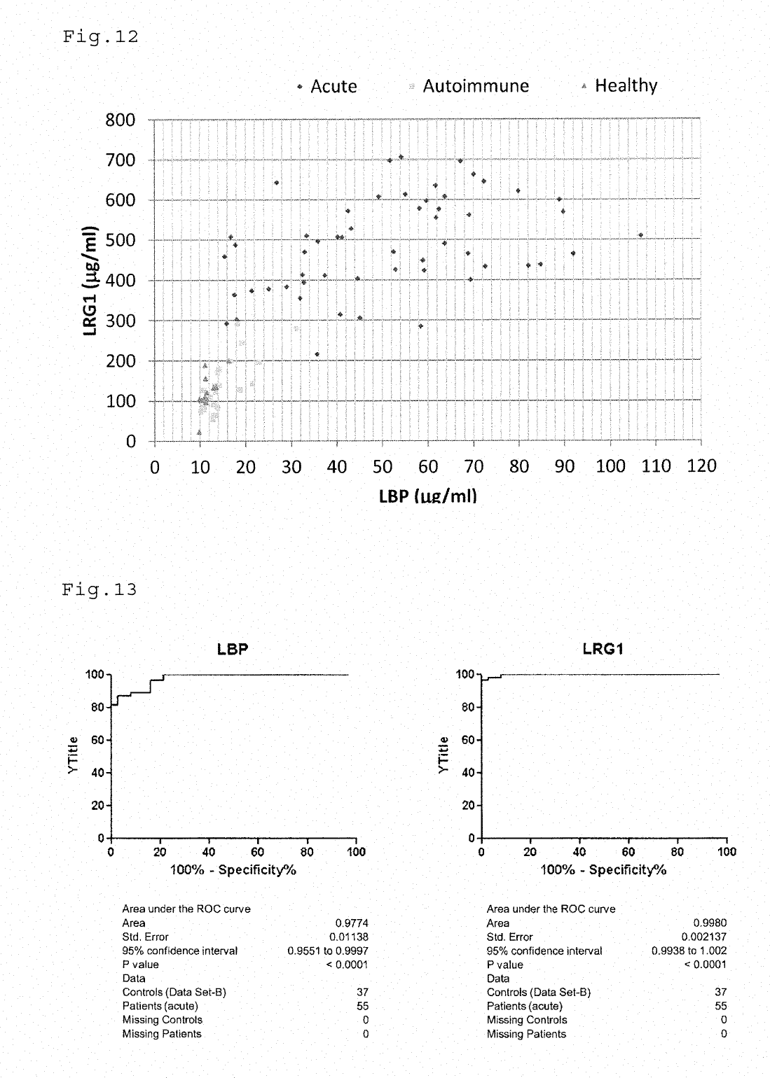

In order to demonstrate the biomarker utility of LBP and LRG1 in the diagnosis of Kawasaki disease (KD), serum LRG1 concentrations (pg/ml) in KD acute phase patients, autoimmune disease patients and healthy infants were plotted on the vertical axis and serum LBP concentrations (μg/ml) on the horizontal axis (FIG. 12). The results revealed that a majority of the 55 KD patients (83.6%; 46 samples) were included in a group showing LRG1 concentrations of 300 μg/ml or more and LBP concentrations of 25 μg/ml or more (excluding autoimmune patients and healthy infants). Therefore, it was found that the two proteins are useful as biomarkers for diagnosing Kawasaki disease.

### Validation of Biomarker Specificity/Sensitivity by ROC Analysis

In order to demonstrate the disease specificity and utility of KD-related proteins LBP and LRG1 in the diagnosis of KD, ROC curves were prepared between KD acute phase and healthy & autoimmune disease (FIG. 13). Diagnostic performance was judged by the magnitude of AUC values. The results revealed that the AUC value of LRG1 was 0.9980 and the AUC value of LBP, 0.9774. Thus, these proteins were shown to be useful as biomarkers for the diagnosis of KD.

Further, cutoff values, sensitivities and specificities were calculated based on the data shown in FIG. 13 (Table 2, lower panel).

Cutoff value (best): the concentration giving a specificity of 95% or more
Cutoff value (better): the concentration giving a specificity of 90% or more
Cutoff value (good): the concentration giving a specificity of 80% or more
Sensitivity: the rate at which a patient who is truly suffering from KD is diagnosed as positive 100%—Specificity: the rate at which a patient suffering from an illness other than KD is misdiagnosed as suffering from KD

TABLE 2
Acute vs autoimmune and healthy

| | LBP | LRG1 |
|---|---|---|
| best | | |
| Specificity (95% or more) | 97.3 | 97.3 |
| Sensitivity % | 87.27 | 98.18 |
| Cutoff value (μg/mL) | 23.98 | 282.1 |
| better | | |
| Specificity (90% or more) | 91.89 | 91.89 |
| Sensitivity % | 89.09 | 100.00 |
| Cutoff value (μg/mL) | 20.31 | 207.70 |
| good | | |
| Specificity (80% or more) | 81.08 | 81.08 |
| Sensitivity % | 96.36 | 100.00 |
| Cutoff value (μg/mL) | 16.54 | 182.70 |

### (Discussion)

Inflammatory proteins such as CRP are found excessively in the sera of patients in the acute phase of KD, and the serum concentrations of these proteins are examined as reference items in blood test. However, many of such inflammatory proteins reflect nonspecific systemic inflammations and do not help in specific differentiation of KD. In the present study making comparison with patients suffering from autoimmune disease (an inflammatory disease like KD) and infants in usual state (i.e., healthy), the inventors have revealed that KD can be diagnosed more specifically by examining the concentrations of both LBP and LRG1 in patients' sera. Since both of these proteins are found in blood at high concentrations, the present inventors believe that a simple and highly precise diagnostic method for Kawasaki disease can be developed by measuring the amounts of these proteins in blood using antibodies specific thereto.

### Example 5

A method of carrying out the present invention by immunochromatography will be described specifically in the following Example. Two types of test strips are preliminarily provided and a sample liquid is applied to a specified site of each test strip.

### Preparation of Test Strip 1a for Immunochromatography

### 1. Preparation of Anti-LBP Polyclonal Antibody

Mice are immunized with an antigen protein (LBP). After feeding for a specified period of time, blood is collected from mice to obtain polyclonal antibodies.

### 2. Immobilization of Anti-LBP Polyclonal Antibodies and Labeled-antibody Specific Antibodies on Membrane 3 (Nitrocellulose Membrane)

Purified anti-LBP polyclonal antibodies are diluted with purified water at a concentration of 1.0 mg/ml to form a suspension (liquid 6) and anti-mouse IgGs polyclonal antibodies are diluted with purified water at a concentration of 1.0 mg/ml to form a suspension (liquid 7); the two suspensions are respectively applied linearly at specified sites on membrane 3 (nitrocellulose membrane) backed with base sheet 2 (PET sheet). The membrane is dried at 45° C. for 30 minutes to obtain an anti-LBP polyclonal antibody/anti-mouse IgGs polyclonal antibody immobilized membrane (hereinafter, designated “antibody immobilized membrane”). This step corresponds to applying liquid 6 to test line (1^{st} detection zone) 6 and liquid 7 to control line (2^{nd} detection zone) 7 in FIG. 14.

### 3. Immobilization of Anti-LBP Polyclonal Antibodies on Colored Polystyrene Particles

Anti-LBP polyclonal antibodies are diluted with purified water at a concentration of 1.0 mg/ml. Colored polystyrene particles are added to the suspension in an amount of 0.1%. The resultant mixture is stirred, followed by addition of carbodiimide in an amount of 1%. The resultant mixture is further stirred. After centrifugation to remove the supernatant, the pellet is resuspended in 50 mM Tris (pH 9.0) with 3% BSA to obtain anti-LBP antibodies bound to colored polystyrene particles (labeled antibodies bound to colored polystyrene particles).

### 4. Application and Drying of Anti-LBP Polyclonal Antibody Binding Colored Polystyrene Particles

The labeled antibodies bound to colored polystyrene particles as obtained in 3 above are applied to a nonwoven fabric of glass fiber in a specified amount of 1.0 μg. The fabric is dried at 45° C. for 30 min to obtain a dry pad (corresponding to conjugate pad 5).

### 5. Lamination of Immobilized Membrane, Dry Pad and Other Components

The antibody immobilized membrane prepared in 2 above (membrane 3 having test line 6 and control line 7) and conjugate pad 5 prepared in 4 above are laminated with other components (i.e., sample pad 4 and absorption pad 8) and cut to a width of 5 mm, making an LBP test strip (test strip 1a).

### Preparation of Test Strip 1b for Immunochromatography

### 1. Preparation of Anti-LRG1 Polyclonal Antibodies

Mice are immunized with an antigen protein (LRG1). After feeding for a specified period of time, blood is collected from mice to obtain polyclonal antibodies.

### 2. Immobilization of Anti-LRG1 Polyclonal Antibodies and Labeled-antibody Specific Antibodies on Membrane 3 (Nitrocellulose Membrane)

Purified anti-LRG1 polyclonal antibodies are diluted with purified water at a concentration of 1.0 mg/ml to form a suspension (liquid 6) and anti-mouse IgGs polyclonal antibodies are diluted with purified water at a concentration of 1.0 mg/ml to form a suspension (liquid 7); the two suspensions are respectively applied linearly at specified sites on membrane 3 (nitrocellulose membrane) backed with base sheet 2 (PET sheet). The membrane is dried at 45° C. for 30 minutes to obtain anti-LRG1 polyclonal antibody/anti-mouse IgGs polyclonal antibody immobilized membrane (hereinafter, designated “antibody immobilized membrane”). This step corresponds to applying liquid 6 to test line (1^{st} detection zone) 6 and liquid 7 to control line (2^{nd} detection zone) 7 in FIG. 14.

### 3. Immobilization of Anti-LRG1 Polyclonal Antibodies on Colored Polystyrene Particles

Anti-LRG1 polyclonal antibodies are diluted with purified water at a concentration of 1.0 mg/ml. Colored polystyrene particles are added to the suspension in an amount of 0.1%. The resultant mixture is stirred, followed by addition of carbodiimide in an amount of 1%. The resultant mixture is further stirred. After centrifugation to remove the supernatant, the pellet is resuspended in 50 mM Tris (pH 9.0) with 3% BSA to obtain anti-LRG1 antibodies bound to colored polystyrene particles (labeled antibodies bound to colored polystyrene particles).

### 4. Application and Drying of Anti-LRG1 Polyclonal Antibody Binding Colored Polystyrene Particles

The labeled antibodies bound to colored polystyrene particles as obtained in 3 above are applied to a nonwoven fabric of glass fiber in a specified amount of 1.0 μg. The fabric is dried at 45° C. for 30 min to obtain a dry pad (corresponding to conjugate pad 5).

### 5. Lamination of Immobilized Membrane, Dry Pad and Other Components

The antibody immobilized membrane prepared in 2 above (membrane 3 having test line 6 and control line 7) and conjugate pad 5 prepared in 4 above are laminated with other components and cut to a width of 5 mm, making an LRG1 test strip (test strip 1b).

### Judging Method

### Step 1

An aliquot of a sample liquid is applied to sample pad 4 of test strip 1a. When color development is observed in both 1^{st} detection zone 6 (test line) and 2^{nd} detection zone 7 (control line), the sample is judged as positive.

### Step 2

An aliquot of the same sample liquid is applied to sample pad 4 of test strip 1b. When color development is observed in both 1^{st} detection zone 6 (test line) and 2^{nd} detection zone 7 (control line), the sample is judged as positive.

### Step 3

When the sample is positive in both step 1 and step 2, it is judged that the subject from whom the sample is derived is likely to be suffering from “Kawasaki disease”.

The above-described Example is for illustrative purposes only, and various modifications can be made without departing from the spirit and scope of the present invention. For example, two polyclonal antibodies used in the above Example may be replaced with two monoclonal antibodies using known methods.

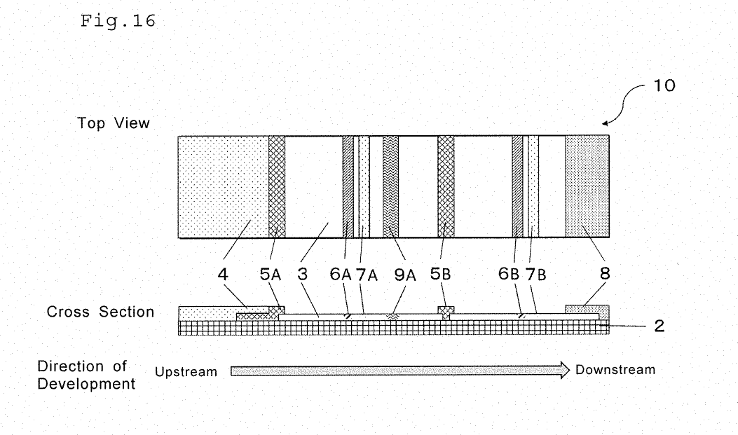

Two types of test strips (1a and 1b) are used in the above Example. Theoretically, it is also possible to allow one test strip to perform two functions. A structure that may be contemplated is shown in FIG. 16. Test strip 10 is roughly divided into two parts; for example, the function of test strip 1 a described above is provided on the upstream side whereas the function of test strip 1b described above is provided on the downstream side. However, in order to ensure that a sample liquid is applied only to sample pad 4 and finally absorbed in absorption pad 8, one sample pad 4 is provided at one end of test strip 10 and one absorption pad 8 at the opposite end. Briefly, sample pad 4 is provided on the upstream side of test strip 10 and absorption pad 8 is provided on the downstream side of test strip 10. The sites involved in antigen-antibody reactions in test strip 1a are provided as 5A, 6A and 7A. The sites involved in antigen-antibody reactions in test strip 1b are provided as 5B, 6B and 7B. In the above explanation, the function of test strip 1a is provided on the upstream side and the function of test strip 1b on the downstream side; however, the locations of these functions may be reversed.

Capturing member 9 has a function of capturing the labeled particles immobilized on 5A. Specifically, antibodies binding to only the labeled particles immobilized on 5A are immobilized at high concentration in capturing member 9. In brief, if, as a result of the sample liquid flowing from upstream to downstream, 5A-derived labeled particles mix with the labeled particles in 5B, the marker function in 6B and 7B may potentially be affected. For preventing this mixing in 5B, it is necessary to capture 5A-derived labeled particles. Therefore, antibodies binding to only the labeled particles are immobilized in capturing member 9A at high concentration. Alternatively, the dimension of capturing member 9 in longitudinal direction (from upstream to downstream) is set at higher values for immobilizing the labeled particles. Specifically, in the former case, the same antibodies as used in 7A are immobilized in 9A at a higher concentration than in 7A (e.g., concentration in 9A is 2 to 10 times higher than that in 7A). In the latter case, the same antibodies as used in 7A are used in 9A at the same concentration but immobilized in a greater length than in 7A (e.g., length of 9A is 2 to 10 times greater than that of 7A). As a method of antibody immobilization, an antibody solution is applied, dripped or sprayed to membrane 3 which is then dried to have the antibodies adsorbed.

Control line 7A is inherently a line for checking whether or not a sample liquid has crossed test line 6A. Since test line 7B also has this function, control line 7A may be omitted.

All publications, patents and patent applications cited herein are incorporated herein by reference in their entirety.

### INDUSTRIAL APPLICABILITY

The present invention is applicable to diagnosis of Kawasaki disease and confirmation of therapeutic effects on the disease.

1. A method detecting a level of at least one component selected from the group consisting of lipopolysaccharide binding protein, leucine-rich alpha-2-glycoprotein, angiotensinogen and retinol binding protein 4, the method comprising
measuring the level of the at least one component selected from the group consisting of lipopolysaccharide binding protein, leucine-rich alpha-2-glycoprotein, angiotensinogen and retinol binding protein 4 in a sample, and
comparing the level of the at least one component selected from the group consisting of lipopolysaccharide binding protein, leucine-rich alpha-2-glycoprotein, angiotensinogen and retinol binding protein 4 measured in the sample to a control sample of the at least one component selected from the group consisting of lipopolysaccharide binding protein, leucine-rich alpha-2-glycoprotein, angiotensinogen and retinol binding protein 4 obtained from a patient suffering from Kawasaki disease, a control sample of the at least one component selected from the group consisting of lipopolysaccharide binding protein, leucine-rich alpha-2-glycoprotein, angiotensinogen and retinol binding protein 4 obtained from a patient not suffering from Kawasaki disease, or a combination of control samples thereof.

2. The method of claim 1, wherein the sample measured is a Kawasaki disease patient.

3. The method of claim 2, wherein the Kawasaki disease patient is receiving treatment for Kawasaki disease.

4. The method of claim 1, wherein the sample is a serum sample or a whole blood sample.

5. The method of claim 1, wherein the measuring comprises measuring with at least one antibody that specifically binds to the at least one component selected from the group consisting of lipopolysaccharide binding protein, leucine-rich alpha-2-glycoprotein, angiotensinogen and retinol binding protein 4.

6. The method of claim 1, wherein the control sample is obtained from a patient suffering from Kawasaki disease.

7. The method of claim 1, wherein the control sample is obtained from a patient not suffering from Kawasaki disease.

8. The method of claim 1, wherein the comparing is to the combination of control samples thereof.

9. The method of claim 3, wherein the sample is obtained between the acute phase and the recovery phase of the Kawasaki disease patient.

10. The method of claim 1, wherein the measuring is performed by immunochromatography.

11. The method of claim 5, wherein the at least one antibody is immobilized on at least one solid support.

12. The method of claim 11, wherein the at least one solid support is at least one test strip.

13. The method of claim 12, wherein the at least one test strip comprises an anti-LBP antibody immobilized carrier, an anti-LRG1 antibody immobilized carrier, or both.

13. The method of claim 12, wherein the measuring comprises two test strips, each comprising different antibodies.

14. The method of claim 12, wherein the measuring comprises a single test strip.

15. A method detecting a level of at least one component selected from the group consisting of lipopolysaccharide binding protein, leucine-rich alpha-2-glycoprotein, angiotensinogen and retinol binding protein 4, the method comprising
comparing the level of the at least one component selected from the group consisting of lipopolysaccharide binding protein, leucine-rich alpha-2-glycoprotein, angiotensinogen and retinol binding protein 4 measured in a sample to a control sample of the at least one component selected from the group consisting of lipopolysaccharide binding protein, leucine-rich alpha-2-glycoprotein, angiotensinogen and retinol binding protein 4 obtained from a patient suffering from Kawasaki disease, a control sample of the at least one component selected from the group consisting of lipopolysaccharide binding protein, leucine-rich alpha-2-glycoprotein, angiotensinogen and retinol binding protein 4 obtained from a patient not suffering from Kawasaki disease, or a combination of control samples thereof.

16. A method of treating Kawasaki disease, comprising treating a subject in need thereof with a Kawasaki disease treatment, the subject having been identified as in need thereof by a method of diagnosing Kawasaki disease comprising measuring a level of at least one component selected from the group consisting of lipopolysaccharide binding protein, leucine-rich alpha-2-glycoprotein, angiotensinogen and retinol binding protein 4.
